(19) United States
(12) Patent Application Publication
(10) Pub. No.: US 2025/0143649 A1
(43) Pub. Date: May 8, 2025

(54) DETECTION AND CHARACTERIZATION OF NEURODEGENERATIVE DISORDER RISK AND SEVERITY

(71) Applicant: Advanced Brain Monitoring, Inc, Carlsbad, CA (US)

(72) Inventors: Daniel J. Levendowski, Carlsbad, CA (US); Vladislav Velimirovic, Belgrade (RS); Bratislav Veljkovic, Belgrade (RS); Nikola Jacimovic, Belgrade (RS); Amy Mathews, Carlsbad, CA (US); Elise Ramos, Tustin, CA (US); Chris Berka, Carlsbad, CA (US)

(21) Appl. No.: 18/939,289

(22) Filed: Nov. 6, 2024

Related U.S. Application Data
(60) Provisional application No. 63/547,521, filed on Nov. 6, 2023.

(51) Int. Cl. A61B 5/00; A61B 5/374; A61B 5/397; G16H 10/60; G16H 15/00

(52) U.S. Cl. CPC A61B 5/7267; A61B 5/374; A61B 5/397; A61B 5/4076; A61B 5/4812; A61B 5/4815; A61B 5/7275; A61B 5/7282; G16H 10/60; G16H 15/00

(57) ABSTRACT

It has proven difficult to automate the detection of neurodegenerative disorders in patients. Thus, detection is currently still performed via visual inspection. Accordingly, embodiments automate the detection of neurodegenerative disorders by deriving sleep biomarker(s) from physiological data, acquired while a subject is sleeping, and applying a classifier to the sleep biomarker(s) to output a risk probability for each neurodegenerative disorder. Embodiments also characterize the neurodegenerative disorder(s) by assigning a risk severity based on the risk probability(ies), output by the classifier.

### CROSS-REFERENCE TO RELATED APPLICATIONS

This application claims priority to U.S. Provisional Patent App. No. 63/547,521, filed on Nov. 6, 2023, which is hereby incorporated herein by reference as if set forth in full.

### BACKGROUND

### Field of the Invention

The embodiments described herein are generally directed to sleep monitoring, and, more particularly, to detecting and characterizing the risk and severity of a neurodegenerative disorder in a subject (e.g., patient), based on physiological data comprising signal patterns representing the subject's sleep architecture.

### Description of the Related Art

The World Health Organization (WHO) has estimated that dementia impacts 50-million individuals worldwide, with an estimated incidence of 10-million new people developing dementia annually, burgeoning to impact an estimated 82-million people by 2030 and 152-million people by 2050 [Ref1]. In the U.S. alone, the healthcare costs associated with neurodegenerative disorders (NDDs) are estimated to exceed $250 billion annually due to population aging [Ref2].

Early diagnosis and targeted intervention are primary goals in dementia care, worldwide [Ref1]. However, available tools that aid in the definition of specific neurodegenerative disorders are currently limited. Reduced beta amyloid and elevated tau proteins in the cerebrospinal fluid predict conversion from mild cognitive impairment (MCI) to Alzheimer's disease dementia (AD) [Ref3, Ref4, Ref5], but are not routinely tested in most memory clinics. In addition, blood-based screening biomarkers, for proteinopathies for the full range of neurodegenerative disorders, still await validation [Ref6].

Sleep impacts brain health by enabling glymphatic clearance, thereby reducing possible toxic metabolites that accumulate during wakefulness [Ref7] and facilitating synaptic homeostasis and consolidation [Ref8, Ref9]. Conversely, insufficient sleep may result in the buildup of beta amyloid, tau, and synuclein proteins, and compromised cognitive function (e.g., memory and learning) [Ref10, Ref11, Ref12, Ref13].

Sleep abnormalities are highly prevalent in patients with neurodegenerative disorders, often appearing in the pre-clinical stage, long before cognitive decline or other objective neurological deficits are detected. The association between sleep disturbances and neurodegeneration may be bidirectional, as sleep disturbances may alternatively cause or result from neurodegenerative processes in the brain [Ref14]. The presence of clinical sleep disorders has been linked with increased risk of future neurodegenerative disorders. For instance, a recent study found patients with primary insomnia, as young adults, had a higher risk of developing dementia than those without primary insomnia [Ref15]. Late-midlife obstructive sleep apnea (OSA) and short sleep duration has been linked to the manifestation of dementia in later life [Ref16].

Studies suggest biomarkers measured during sleep hold promise in the characterization and monitoring of neurodegeneration along the continuum of prodromal disease, symptomatic mild cognitive impairment, and eventual dementia.

Decreased sleep spindle oscillations during non-rapid eye movement (NREM) sleep have been associated with cognitive decline in older adults, increased tau levels, and development of dementia in patients with Parkinson's disease [Ref17]. Patients with AD and progressive supranuclear palsy (PSP) also exhibit reduced spindle activity, reflecting decreased thalamocortical network neuronal activity [Ref18]. Reduced slow-wave sleep (SWS or N3) has been associated with increased beta amyloid concentrations in cerebrospinal fluid [Ref19, Ref20]. Sleep architectural features are also linked to increased risk of future neurodegenerative disorders. These features include prolonged sleep latency, reduced sleep efficiency, and REM sleep impairment [Ref21, Ref22]. Autonomic nervous system dysfunction that manifests in α-synucleinopathy related Lewy body disease (LBD) including Parkinson's disease (PD), dementia with Lewy bodies (DLB), multiple system atrophy (MSA), and the like, may occur in any stage of the disease, and may precede or follow other characteristic motor or cognitive symptoms [Ref23, Ref24]. Idiopathic rapid eye movement (REM) sleep behavioral disorder (iRBD) has been established as a prodromal synucleinopathy (pSYN) in most adults, and an important predictor of PD and DLB [Ref25, Ref26]. Approximately, 70-75% of those diagnosed with iRBD undergo phenoconversion within a 10-year to 15-year period, with approximately 50% developing a parkinsonism-predominant syndrome (most often PD), and the others developing a dementia-predominant syndromes [Ref28]. Patients with Parkinson's disease and RBD appear to represent a distinct phenotype with more severe cognitive and depressive symptoms, and a faster rate of disease progression [Ref29-Ref31].

Investigations have uncovered a number of new sleep biomarkers associated with specific NDD phenotypes. For example, sleeping more than two hours per night in the supine position originally demonstrated a strong association in patients predominantly with MCI and AD [Ref32]. A follow-up study now shows that LBD, AD, and MCI patients all exhibit greater supine sleep time than those with normal cognition (CG) [Ref33]. Electroencephalogram (EEG) slowing, measured by the ratio of theta-wave power to alpha-wave power during NREM sleep, was found to be significantly greater in patients with AD and MCI [Ref34]. Increased EEG slowing was also a featured characteristic of atypical N3 (AN3) sleep, when delta-wave power relative to theta-wave power and sigma-wave power relative to alpha-wave power were aberrant. AN3 sleep was found to be significantly greater in patients with LBD compared to MCI, iRBD, or CG [Ref35]. Consistently elevated electromyographic (EMG) activity relative to delta-wave power and theta-wave power is the hallmark of the novel sleep biomarker, NREM hypertonia (NRH). The prevalence of NREM hypertonia was greater in patients with LBD, PSP, PD, and iRBD, as compared to AD, MCI, and CG [Ref36, Ref37]. NRH was found to be strongly associated with REM sleep without atonia (RSWA), the confirmatory biomarker for RBD [Ref38]. Patients with PSP, LBD, iRBD, AD, and CG were found to exhibit unique patterns when NREM hypertonia and spindle duration were combined based on normal and/or abnormal characterizations [Ref39]. Blunted heart-rate variability associated with autonomic dysfunction in patients with LBD was observed using the pulse-rate derived autonomic activation index (AAI) [Ref40]. The associations between AAI, NREM hypertonia, and sleep spindle duration were limited, suggesting that each of these three sleep biomarkers may independently differentiate LBD, PD, and iRBD from ADem and CG. Sleep oscillations during NREM sleep have been associated with AD [Ref41].

Early characterization of the underlying NDD phenotype (e.g., AD vs. LBD) may be important in the selection of an appropriate intervention to help delay dementia onset. In the early stages of memory decline, it may be difficult to determine if an MCI patient will ultimately develop an AD, LBD, or mixed dementia pathology. As a result, the NDD phenotype is commonly established because of dementia-specific symptoms, and a subject's NDD phenotype may be misdiagnosed based on their dementia-like symptoms. In PD patients, only 50% exhibit the prodromal synucleinopathy biomarker (i.e., RBD) associated with future phenoconversion to dementia. The balance of PD patients are affected by PD-related symptoms (e.g., tremors, etc.) with normal age-related cognitive decline. Thus, MCI and PD patients will exhibit greater homogeneity in abnormal sleep characteristics, and the rate of change in these characteristics will be individual-specific.

When surveyed, over 80% adults felt it was important to know their risk for a neurodegenerative disorder even if nothing may currently be done to prevent or cure the neurodegenerative disorder. In particular, subjects, identified at risk of a neurodegenerative disorder, would use the information to help plan for the future, and would seek more information about medications and adaptive therapies designed to delay onset [Ref42, Ref43].

In summary, specific sleep biomarkers may be used to characterize NDD phenotypes, and individuals want to have access to this information in order to monitor the disease trajectory.

### SUMMARY

Accordingly, systems, methods, and non-transitory computer-readable media are disclosed for detecting and characterizing NDD risk and severity in a subject.

In an embodiment, a method of characterizing a neurodegenerative disorder (NDD) comprises using at least one hardware processor to: acquire physiological data for a subject, wherein the physiological data are obtained while the subject is sleeping; derive one or more sleep biomarkers based on the physiological data; apply a classifier to the one or more sleep biomarkers, wherein the classifier outputs a risk probability for each of one or more neurodegenerative disorders; assign a risk severity based on the risk probability for each of the one or more neurodegenerative disorders; and generate a report that indicates the risk severity for the subject.

The one or more sleep biomarkers may comprise a measure of time spent in rapid eye movement (REM) sleep.

The one or more sleep biomarkers may comprise a measure of spindle activity. Deriving the measure of spindle activity may comprise: detecting each sleep spindle by identifying a spindle peak comprising a burst in both sigma-wave power and alpha-wave power, within the physiological data, determining a start time and an end time of the sleep spindle around the spindle peak based on at least one first threshold, determining a duration of the sleep spindle based on the start time and the end time, and detecting the sleep spindle when the duration of the sleep spindle satisfies at least one second threshold; and computing the measure of spindle activity based on the duration of each detected sleep spindle.

The one or more sleep biomarkers may comprise a measure of atypical slow-wave (AN3) sleep.

The one or more sleep biomarkers may comprise a measure of non-rapid eye movement (NREM) hypertonia (NRH). Deriving the measure of NREM hypertonia comprises automatically detecting one or more episodes of NREM hypertonia by: for each of a plurality of epochs, represented in the physiological data, determining whether or not the epoch exhibits an abnormal sleep characteristic based on signals, in the physiological data, representing delta-wave power without ocular activity, theta-wave power, sigma-wave power, and electromyographic (EMG) power, and determining whether or not a standard deviation of the EMG power satisfies a predefined threshold within a set of two or more epochs that includes the epoch; connecting two or more of the plurality of epochs, that each exhibits the abnormal sleep characteristic and for which the standard deviation of the EMG power satisfies the predefined threshold within the set of two or more epochs that includes the epoch, into an NRH block; extending one or more NRH blocks to include one or more surrounding epochs; and excluding any NRH block that satisfies one or more exclusion criteria, wherein the one or more episodes of NREM hypertonia consist of any non-excluded NRH blocks. Determining whether or not the epoch exhibits an abnormal sleep characteristic may comprise: determining that the epoch does not exhibit the abnormal sleep characteristic when the delta-wave power without ocular activity exceeds a first threshold; calculating a delta threshold based on a theta-EMG ratio of the theta-wave power and the EMG power; calculating a theta threshold based on a delta-EMG ratio of the delta-wave power without ocular activity and the EMG power; determining whether or not the delta-EMG ratio is within a first range based on the delta threshold; determining whether or not the theta-EMG ratio is within a second range based on the theta threshold; determining whether or not a sigma-EMG ratio of the sigma-wave power and the EMG power is within a third range; determining that the epoch does not exhibit the abnormal sleep characteristic when either the delta-EMG ratio is not within the first range, the theta-EMG ratio is not within the second range, or the sigma-EMG ratio is not within the third range; and determining that the epoch exhibits the abnormal sleep characteristic when the delta-EMG ratio is within the first range, the theta-EMG ratio is within the second range, and the sigma-EMG ratio is within the third range. The one or more exclusion criteria may comprise one or more of a presence of the NRH block within a predefined time duration immediately following sleep onset, the presence of the NRH block within a predefined time duration of uprightness, or the NRH block corresponding in time to a skin-electrode impedance greater than a predefined threshold impedance.

The one or more sleep biomarkers may comprise a measure of autonomic activation.

The one or more sleep biomarkers may comprise relative theta-wave power.

The one or more sleep biomarkers may comprise a ratio of theta-wave power to alpha-wave power.

The one or more sleep biomarkers may comprise a measure of sleep efficiency.

The one or more sleep biomarkers may comprise sleep duration in a supine position.

The one or more sleep biomarkers may comprise a measure of electroencephalographic (EEG) slowing.

The one or more sleep biomarkers may comprise a measure of rapid eye movement (REM) sleep without atonia (RSWA) events. Deriving the measure of RSWA events may comprise: detecting the RSWA events by, for each of a plurality of epochs, represented in the physiological data, filtering an electromyographic (EMG) signal in the physiological data for the epoch, extracting a measure of EMG power from the filtered EMG signal, computing baseline EMG power during REM sleep, and detecting the RSWA event based on the measure of EMG power and the baseline EMG power; and computing the measure of RSWA events based on the detected RSWA events.

The one or more sleep biomarkers may comprise a pattern of oscillatory events.

The one or more sleep biomarkers may be derived further based on a health record of the subject.

The one or more neurodegenerative disorders may be a plurality of neurodegenerative disorders. The report may comprise the risk severity, a two-way comparison of each pair of the plurality of neurodegenerative disorders, and an analysis of each of the one or more sleep biomarkers.

The one or more sleep biomarkers may be a plurality of sleep biomarkers.

It should be understood that any of the features in the methods above may be implemented individually or with any subset of the other features in any combination. Thus, to the extent that the appended claims would suggest particular dependencies between features, disclosed embodiments are not limited to these particular dependencies. Rather, any of the features described herein may be combined with any other feature described herein, or implemented without any one or more other features described herein, in any combination of features whatsoever. In addition, any of the methods, described above and elsewhere herein, may be embodied, individually or in any combination, in executable software modules of a processor-based system, such as a server, and/or in executable instructions stored in a non-transitory computer-readable medium.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 illustrates an example infrastructure, in which one or more of the processes described herein may be implemented, according to an embodiment;

FIG. 2 illustrates an example processing system, by which one or more of the processes described herein may be executed, according to an embodiment;

FIG. 3 illustrates a process for detecting a neurodegenerative disorder, according to an embodiment;

FIG. 4 illustrates an exemplary set of rules for analyzing risk probabilities to determine an overall risk severity, according to an embodiment;

FIG. 5 illustrates a screen of a graphical user interface, comprising a report of the overall risk severity, according to an embodiment;

FIG. 6 illustrates a process for deriving the measure of spindle activity, according to an embodiment;

FIG. 7 illustrates a process for deriving episode(s) of NREM hypertonia, according to an embodiment;

FIG. 8 illustrates a process for deriving a measure of RSWA events, according to an embodiment;

FIGS. 9A-9J illustrate examples of the proportion of subjects, classified into each neurodegenerative disorder, identified by individual sleep biomarkers, as well as the distribution of ages of the subjects, according to an embodiment;

FIG. 10 illustrates an example set of sleep signals from a subject that may be used to derive one or more sleep biomarkers, according to an embodiment;

FIG. 11 illustrates an example set of sleep signals from a subject that may be used to characterize REM sleep without atonia (RSWA), according to an embodiment;

FIG. 12 illustrates an example set of sleep signals from a subject that highlights the signal patterns that are unique to the slow waves that appear during N3 sleep versus those that appear during AN3 sleep, according to an embodiment; and

FIG. 13 illustrates an example EMG signal, before and after filtering, according to an embodiment, according to an embodiment.

### DETAILED DESCRIPTION

In an embodiment, systems, methods, and non-transitory computer-readable media are disclosed for detecting and characterizing NDD risk and severity in a subject. After reading this description, it will become apparent to one skilled in the art how to implement the invention in various alternative embodiments and alternative applications. However, although various embodiments of the present invention will be described herein, it is understood that these embodiments are presented by way of example and illustration only, and not limitation. As such, this detailed description of various embodiments should not be construed to limit the scope or breadth of the present invention as set forth in the appended claims.

### 1. INFRASTRUCTURE

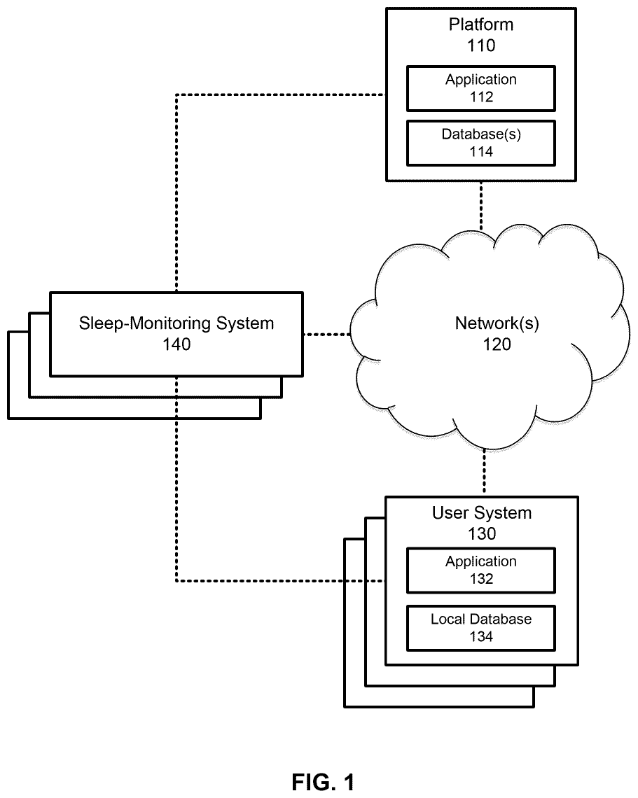

FIG. 1 illustrates an example infrastructure in which one or more of the disclosed processes may be implemented, according to an embodiment. The infrastructure may comprise a platform 110 (e.g., one or more servers) which hosts and/or executes one or more of the various processes, methods, functions, and/or software modules described herein. Platform 110 may comprise dedicated servers, or may instead be implemented in a computing cloud, in which the resources of one or more servers are dynamically and elastically allocated to multiple tenants based on demand. In either case, the servers may be collocated and/or geographically distributed. Platform 110 may also comprise or be communicatively connected to a server application 112 and/or one or more databases 114.

In addition, platform 110 may be communicatively connected to one or more user systems 130 via one or more networks 120. Platform 110 may also be communicatively connected to one or more sleep-monitoring systems 140 via one or more networks 120. Alternatively, one or more sleep-monitoring systems 140 may be directly connected to platform 110 and/or directly connected to a user system 130.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or sleep-monitoring systems 140 via the Internet, but may be connected to one or more other user systems 130 and/or sleep-monitoring systems 140 via an intranet. Furthermore, while only a few user systems 130 and sleep-monitoring systems 140, one server application 112, and one set of database(s) 114 are illustrated, it should be understood that the infrastructure may comprise any number of user systems, sleep-monitoring systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, and/or the like. However, it is generally contemplated that user system 130 would comprise the personal computer or professional workstation of a subject (e.g., human patient), physician, health technician, researcher, data scientist, or other individual involved in a sleep study or associated with a healthcare provider of the subject. Each user system 130 may comprise or be communicatively connected to a client application 132 and/or one or more local databases 134.

Sleep-monitoring system(s) 140 may comprise any device or system of devices configured to acquire or collect physiological data for a subject while the subject sleeps. Each sleep-monitoring system 140 may comprise one or more sensors that are attachable and detachable from one or more body parts of the subject (e.g., head, neck, chin, chest, arm, leg, etc.). These sensors may include, for example, electroencephalographic (EEG) sensor(s), electromyographic (EMG) sensor(s), electrocardiogramsor(s), skin electrode(s), a blood pressure monitor, heart rate monitor, thermometer(s), a pulse oximeter, and/or the like. Sleep-monitoring system 140 may collect the output signals from all of the sensor(s) as physiological data for the subject, and transmit the physiological data to platform 110 (e.g., via network(s) 120 or directly) and/or user system 130 (e.g., via network(s) 120 or directly). Sleep-monitoring system 140 may comprise or consist of a compact device that is worn by the subject (e.g., on the subject's head). Alternatively, sleep-monitoring system 140 may be a non-wearable device or set of devices used in conventional sleep studies.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. It should be understood that platform 110 may also respond to other requests from user system(s) 130.

Platform 110 may comprise, be communicatively coupled with, or otherwise have access to one or more database(s) 114. For example, platform 110 may comprise one or more database servers which manage one or more databases 114. Server application 112 executing on platform 110 and/or client application 132 executing on user system 130 may submit data (e.g., user data, form data, etc.) to be stored in database(s) 114, and/or request access to data stored in database(s) 114. Any suitable database may be utilized, including without limitation MySQL™, Oracle™, IBM™, Microsoft SQL™, Access™, PostgreSQL™, MongoDB™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from user system(s) 130 and/or sleep-monitoring system(s) 140, and provide responses in extensible Markup Language (XML), JavaScript Object Notation (JSON), and/or any other suitable or desired format. In such embodiments, platform 110 may provide an application programming interface (API) which defines the manner in which user system(s) 130 and/or sleep-monitoring system(s) 140 may interact with the web service. Thus, user system(s) 130 and/or sleep-monitoring system(s) 140 (which may themselves comprise servers), can define their own user interfaces, and rely on the web service to implement or otherwise provide the backend processes, methods, functionality, storage, and/or the like, described herein. For example, in such an embodiment, a client application 132, executing on one or more user system(s) 130, may interact with a server application 112 executing on platform 110 to execute one or more or a portion of one or more of the various functions, processes, methods, and/or software modules described herein.

Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the software described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules comprising instructions that implement one or more of the processes, methods, or functions described herein.

### 2. EXAMPLE PROCESSING SYSTEM

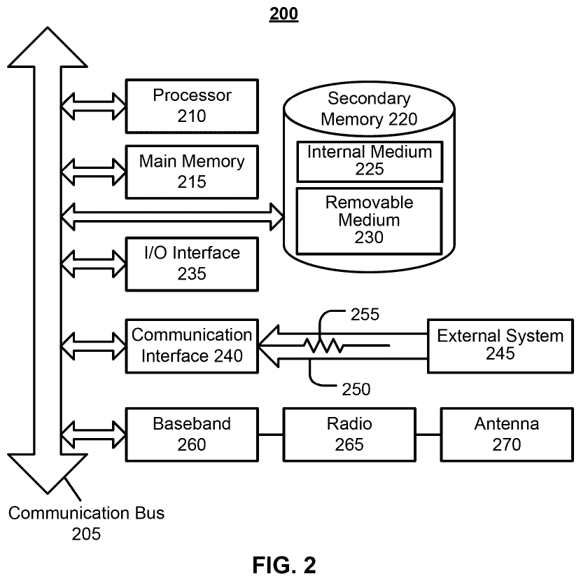

FIG. 2 illustrates an example processing system 200, by which one or more of the processes described herein may be executed, according to an embodiment. For example, system 200 may be used as or in conjunction with one or more of the processes, methods, or functions (e.g., to store and/or execute the software) described herein, and may represent components of platform 110, user system(s) 130, sleep-monitoring system(s) 140, and/or other processing devices described herein. System 200 can be any processor-enabled device (e.g., server, personal computer, etc.) that is capable of wired or wireless data communication. Other processing systems and/or architectures may also be used, as will be clear to those skilled in the art.

System 200 may comprise one or more processors 210. Processor(s) 210 may comprise a central processing unit (CPU). Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a subordinate processor (e.g., back-end processor), an additional microprocessor or controller for dual or multiple processor systems, and/or a coprocessor. Such auxiliary processors may be discrete processors or may be integrated with a main processor 210. Examples of processors which may be used with system 200 include, without limitation, any of the processors (e.g., Pentium™, Core i7™, Core i9™, Xeon™, etc.) available from Intel Corporation of Santa Clara, California, any of the processors available from Advanced Micro Devices, Incorporated (AMD) of Santa Clara, California, any of the processors (e.g., A series, M series, etc.) available from Apple Inc. of Cupertino, any of the processors (e.g., Exynos™) available from Samsung Electronics Co., Ltd., of Seoul, South Korea, any of the processors available from NXP Semiconductors N.V. of Eindhoven, Netherlands, and/or the like.

Processor(s) 210 may be connected to a communication bus 205. Communication bus 205 may include a data channel for facilitating information transfer between storage and other peripheral components of system 200. Furthermore, communication bus 205 may provide a set of signals used for communication with processor 210, including a data bus, address bus, and/or control bus (not shown). Communication bus 205 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 200 may comprise main memory 215. Main memory 215 provides storage of instructions and data for programs executing on processor 210, such as any of the software discussed herein. It should be understood that programs stored in the memory and executed by processor 210 may be written and/or compiled according to any suitable language, including without limitation C/C++, Java, JavaScript, Perl, Python, Visual Basic, .NET, and the like. Main memory 215 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

System 200 may comprise secondary memory 220. Secondary memory 220 is a non-transitory computer-readable medium having computer-executable code and/or other data (e.g., any of the software disclosed herein) stored thereon. In this description, the term “computer-readable medium” is used to refer to any non-transitory computer-readable storage media used to provide computer-executable code and/or other data to or within system 200. The computer software stored on secondary memory 220 is read into main memory 215 for execution by processor 210. Secondary memory 220 may include, for example, semiconductor-based memory, such as programmable read-only memory (PROM), erasable programmable read-only memory (EPROM), electrically erasable read-only memory (EEPROM), and flash memory (block-oriented memory similar to EEPROM).

Secondary memory 220 may include an internal medium 225 and/or a removable medium 230. Internal medium 225 and removable medium 230 are read from and/or written to in any well-known manner. Internal medium 225 may comprise one or more hard disk drives, solid state drives, and/or the like. Removable storage medium 230 may be, for example, a magnetic tape drive, a compact disc (CD) drive, a digital versatile disc (DVD) drive, other optical drive, a flash memory drive or card, and/or the like.

System 200 may comprise an input/output (I/O) interface 235. I/O interface 235 provides an interface between one or more components of system 200 and one or more input and/or output devices. Example input devices include, without limitation, sensors, keyboards, touch screens or other touch-sensitive devices, cameras, biometric sensing devices, computer mice, trackballs, pen-based pointing devices, and/or the like. Examples of output devices include, without limitation, other processing systems, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet computer, or other mobile device).

System 200 may comprise a communication interface 240. Communication interface 240 allows software to be transferred between system 200 and external devices (e.g. printers), networks, or other information sources. For example, computer-executable code and/or data may be transferred to system 200 from a network server (e.g., platform 110) via communication interface 240. Examples of communication interface 240 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 200 with a network (e.g., network(s) 120) or another computing device. Communication interface 240 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software transferred via communication interface 240 is generally in the form of electrical communication signals 255. These signals 255 may be provided to communication interface 240 via a communication channel 250 between communication interface 240 and an external system 245 (e.g., which may correspond to a sleep-monitoring system 140, an external computer-readable medium, and/or the like). In an embodiment, communication channel 250 may be a wired or wireless network (e.g., network(s) 120), or any variety of other communication links. Communication channel 250 carries signals 255 and can be implemented using a variety of wired or wireless communication means including wire or cable, fiber optics, conventional phone line, cellular phone link, wireless data communication link, radio frequency (“RF”) link, or infrared link, just to name a few.

Computer-executable code is stored in main memory 215 and/or secondary memory 220. Computer-executable code can also be received from an external system 245 via communication interface 240 and stored in main memory 215 and/or secondary memory 220. Such computer-executable code, when executed, may enable system 200 to perform the various functions of the disclosed embodiments as described elsewhere herein.

In an embodiment that is implemented using software, the software may be stored on a computer-readable medium and initially loaded into system 200 by way of removable medium 230, I/O interface 235, or communication interface 240. In such an embodiment, the software is loaded into system 200 in the form of electrical communication signals 255. The software, when executed by processor 210, preferably causes processor 210 to perform one or more of the processes and functions described elsewhere herein.

System 200 may comprise wireless communication components that facilitate wireless communication over a voice network and/or a data network (e.g., in the case of user system 130). The wireless communication components comprise an antenna system 270, a radio system 265, and a baseband system 260. In system 200, radio frequency (RF) signals are transmitted and received over the air by antenna system 270 under the management of radio system 265.

In an embodiment, antenna system 270 may comprise one or more antennae and one or more multiplexors (not shown) that perform a switching function to provide antenna system 270 with transmit and receive signal paths. In the receive path, received RF signals can be coupled from a multiplexor to a low noise amplifier (not shown) that amplifies the received RF signal and sends the amplified signal to radio system 265.

In an alternative embodiment, radio system 265 may comprise one or more radios that are configured to communicate over various frequencies. In an embodiment, radio system 265 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 265 to baseband system 260.

If the received signal contains audio information, then baseband system 260 decodes the signal and converts it to an analog signal. Then the signal is amplified and sent to a speaker. Baseband system 260 also receives analog audio signals from a microphone. These analog audio signals are converted to digital signals and encoded by baseband system 260. Baseband system 260 also encodes the digital signals for transmission and generates a baseband transmit audio signal that is routed to the modulator portion of radio system 265. The modulator mixes the baseband transmit audio signal with an RF carrier signal, generating an RF transmit signal that is routed to antenna system 270 and may pass through a power amplifier (not shown). The power amplifier amplifies the RF transmit signal and routes it to antenna system 270, where the signal is switched to the antenna port for transmission.

Baseband system 260 is communicatively coupled with processor(s) 210, which have access to memory 215 and 220. Thus, software can be received from baseband processor 260 and stored in main memory 210 or in secondary memory 220, or executed upon receipt. Such software, when executed, can enable system 200 to perform the various functions of the disclosed embodiments.

### 3. DETECTION OF NEURODEGENERATIVE DISORDERS

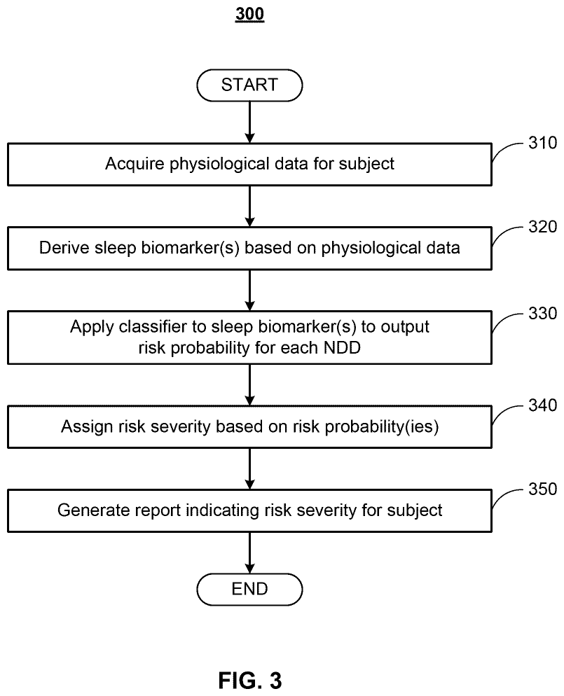

FIG. 3 illustrates a process 300 for detecting a neurodegenerative disorder, according to an embodiment. Process 300 may be implemented by server application 112, client application 132, sleep-monitoring system 140, a combination of server application 112 and client application 132, a combination of sleep-monitoring system 140 and server application 112, a combination of sleep-monitoring system 140 and client application 132, or a combination of sleep-monitoring system 140, server application 112, and client application 132. While process 300 is illustrated with a certain arrangement and ordering of subprocesses, process 300 may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

Subprocess 310 may acquire physiological data for a subject. The physiological data may be obtained while the subject is sleeping. The physiological data may comprise information for one or a plurality of signals acquired while the subject is sleeping, such as one or more characteristics (e.g., frequency, amplitude or power, waveform, etc.) of brain waves obtained through electroencephalography (EEG). These brain waves may include, without limitation, alpha waves, beta waves, delta waves, theta waves, sigma waves, gamma waves, mu waves, K-complexes, vertex sharp waves, epsilon waves, and/or the like. The physiological data may also comprise information, such as an electrocardiogramal, electromyographic signal, skin-electrode impedance, apnoea hypopnea index, blood pressure, body temperature, skin temperature, airway pressure, photoplethysmographic signal, electrooculographic signal, pulse, heart rate, heart-rate variability, impedance pneumographic signal, oxygen desaturation index, blood oxygen saturation, and/or the like.

Subprocess 310 may also acquire other information for the subject, in addition to the physiological data. This other information may comprise the health record (e.g., electronic health record) of the subject. The health record may comprise demographic information (e.g., age, sex, etc.), anthropomorphic information (e.g., neck size, weight, etc.), comorbidities (e.g., depression, sleep apnea, etc.), medications (e.g., anti-depressants, sedatives, etc.), questionnaire or test results (e.g., Mini-Mental State Examination (MMSE), Montreal Cognitive Assessment (MoCA), amnestic vs. non-amnestic mild cognitive impairment (MCI), dream-enactment behavior, etc.), and/or the like. Such information may influence the physiological data.

Subprocess 320 may derive one or more, and generally a plurality, of sleep biomarkers based on the physiological data, received in subprocess 310. These sleep biomarker(s) may include or be based on, without limitation, overall sleep characteristics (e.g., sleep efficiency, sleep position, autonomic activation, proportion of normal sleep, proportion of abnormal sleep), NREM sleep characteristics (e.g., NREM hypertonia, sleep spindle activity, depth of sleep, amplitude and frequency of slow waves, detection of abnormal slow-wave activity, relationships between slow waves and spindle activity, etc.), REM sleep characteristics (e.g., EMG artifact intrusion into EEG signal, detection of phasic and/or tonic EMG excursions, relationships between slow waves and spindle activity, amplitude of phasic activity, differentiation of phasic and tonic periods, etc.), and/or the like. The sleep biomarker(s) may be derived further based on the other information acquired for the subject, such as the subject's health record, including demographic information, anthropomorphic information, comorbidities, medications, questionnaire or test results, and/or the like. It should be understood that the types of information utilized to derive the sleep biomarker(s) may be reduced or expanded, from those specifically described herein, to include any variables that may be useful for characterizing a neurodegenerative disorder and/or assessing the risk of a neurodegenerative disorder.

Specific sleep biomarkers will be described in greater detail elsewhere herein. In general, the sleep biomarker(s) should be selected for group differentiation between different neurodegenerative disorders. In other words, a combination of sleep biomarkers should be capable of differentiating between each of a plurality of neurodegenerative disorders. Ideally, each sleep biomarker provides diagnostic capabilities with a high degree of sensitivity, specificity, and reliability. In an embodiment, the sleep biomarker(s) are selected to characterize features that are unique to any or several target neurodegenerative disorders, while being relatively independent of each other, such that each sleep biomarker has a low degree of cross-correlation with the other sleep biomarker(s). The table below highlights the capabilities, of a plurality of exemplary sleep biomarkers, to distinguish between specific neurodegenerative disorders via the application of thresholds that define abnormality:

| | | | | | |
|---|---|---|---|---|---|
| Sleep | Diseased NDD | Uninfected | ROC AUC | | |
| Biomarker | Category(s) | Category(s) | (95% CI) | Sensitivity | Specificity |

Lewy Body Dementia

| | | | | | |
|---|---|---|---|---|---|
| REM Time | LBD | AD, PD, MCI, | 0.78 | 0.63 | 0.93 |
| | | iRBD, CG | (0.67-0.90) | | |
| Spindle | LBD | AD, PD, MCI, | 0.74 | 0.84 | 0.63 |
| Duration | | iRBD, CG | (0.64-0.83) | | |
| Atypical N3 | LBD | AD, PD, MCI, | 0.66 | 0.42 | 0.89 |
| | | iRBD, CG | (0.54-0.77) | | |

Synucleinopathy

| | | | | | |
|---|---|---|---|---|---|
| Non-REM | LBD, PD, iRBD | AD, MCI, CG | 0.74 | 0.70 | 0.79 |
| Hypertonia | | | (0.67-0.82) | | |
| Autonomic | LBD, PD, iRBD | AD, MCI, CG | 0.65 | 0.54 | 0.75 |
| Activation | | | (0.57-0.73) | | |

Dementia or Cognitive Impairment

| | | | | | |
|---|---|---|---|---|---|
| Relative Theta | LBD, AD, MCI | PD, iRBD, CG | 0.71 | 0.68 | 0.73 |
| | | | (0.64-0.77) | | |
| Theta/Alpha | LBD, AD, MCI | PD, iRBD, CG | 0.63 | 0.52 | 0.74 |
| Ratio | | | (0.56-0.70) | | |

NDD Condition

| | | | | | |
|---|---|---|---|---|---|
| Sleep | LBD, AD, MCI, | CG | 0.65 | 0.41 | 0.89 |
| Efficiency | PD, iRBD | | (0.59-0.71) | | |
| Supine Sleep | LBD, AD, MCI, | CG | 0.61 | 0.63 | 0.59 |
| | PD, iRBD | | (0.54-0.69) | | |

Subprocess 330 may apply a classifier to the sleep biomarker(s), derived in subprocess 320. The classifier may output a risk probability for each of a plurality of neurodegenerative disorders. For example, a feature vector, comprising the value of each sleep biomarker, may be input to the classifier, and the classifier may output a vector comprising the risk probability of each of one or more, and generally a plurality, of neurodegenerative disorders. The output vector may also comprise a risk probability of no neurodegenerative disorders. In other words, the classifier may output a risk probability for each of a plurality of classes that comprises or consists of one or a plurality of NDD classes and a normal class. The sum of all of the risk probabilities in the output vector may sum to one (or one-hundred percent). The classifier will be described in greater detail elsewhere herein.

It should be understood that the risk probability of the normal class represents a probability that the subject does not have any of the neurodegenerative disorder(s) supported by the classifier. In other words, the probability is not strictly speaking a probability of a “risk.” It might be more accurate to say that the probability of the normal class is a non-risk probability. However, for simplicity of description, each output of the classifier will be referred to herein as a “risk probability,” even in the context of the normal class.

Subprocess 340 may assign a risk severity based on the risk probability(ies), output by the classifier, in subprocess 330, for the one or more neurodegenerative disorders. In particular, subprocess 340 may analyze the risk probability(ies) in the output vector of the classifier, to determine the risk severity. This analysis may comprise comparing one or more of the risk probabilities, and/or mathematical variables computed from one or more risk probabilities, to respective thresholds. Ideally, the analysis is designed to minimize the likelihood of false positives (i.e., classifying a normal subject as abnormal), and ensure that all possible permutations of risk probabilities can be assigned to an overall risk severity.

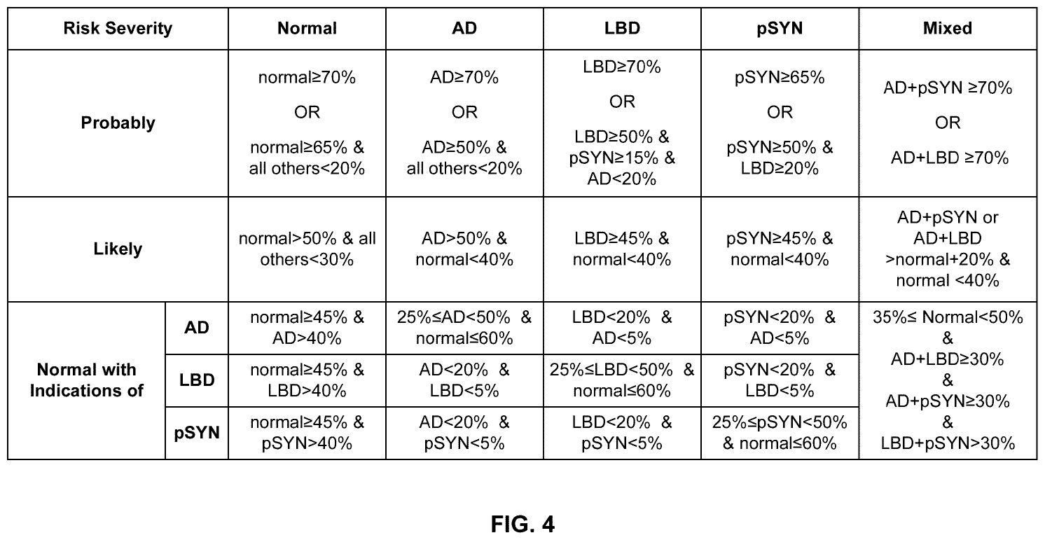

FIG. 4 illustrates an exemplary set of rules for analyzing the risk probabilities to assign a risk severity, according to an embodiment of subprocess 340. It should be understood that the precise rules will depend on the neurodegenerative disorder(s) that are selected. In this example, there are a plurality of neurodegenerative disorders comprising Alzheimer's disease dementia (AD), Lewy body disease (LBD), and prodromal synucleinopathy (pSYN) (e.g., which may be defined based on iRBD), and the classifications of risk severity include probably, likely, and normal with indications. The rules determine the risk severity of each of these neurodegenerative disorders, as well as the likelihood that none of the plurality of neurodegenerative disorders are present (i.e., normal), and the risk severity of the presence of more than one neurodegenerative disorder (i.e., mixed). The mixed class represents that the combination of risk probabilities across all neurodegenerative disorders is sufficient to report an abnormality, but no definitive neurodegenerative disorder can be specified. The risk severity of “normal with indications” of one of the neurodegenerative disorders suggests an increased risk of a neurodegenerative disorder, even though the subject is within normal ranges. The risk severity of “likely” typically suggests a 50% or greater probability of the respective class, and the risk severity of “probably” typically suggests a probability of the respective class that is trending towards 100%. The table below provides some exemplary applications of the illustrated set of rules given exemplary risk probabilities output by the classifier for each of the neurodegenerative disorders and the normal class.

| Risk Probabilities (%) | | | | |
|---|---|---|---|---|
| Normal | AD | LBD | pSYN | Overall Risk Severity |
| 73% | 24% | 0% | 3% | probably normal |
| 64% | 17% | 12% | 7% | likely normal |
| 50% | 43% | 0% | 7% | likely normal with indications of AD |
| 57% | 21% | 3% | 19% | likely normal with mixed indications |
| 35% | 4% | 7% | 54% | likely pSYN |
| 31% | 23% | 10% | 36% | likely mixed |
| 15% | 76% | 0% | 9% | probably AD |
| 10% | 16% | 74% | 0% | probably LBD |
| 13% | 47% | 36% | 4% | probably mixed AD/LBD |
| 4% | 30% | 66% | 0% | probably mixed LBD/AD |

Subprocess 350 may generate a report that indicates the risk severity, assigned in subprocess 340, for the subject. Subprocess 350 may comprise generating a report that comprises the risk severity, a comparison of each pair of a plurality of neurodegenerative disorders (i.e., if the classifier supports a plurality of neurodegenerative disorders), and/or an analysis of each of at least a subset of the sleep biomarker(s). The report may be provided to a user, in order to notify the user of the risk severity. The user may be the subject, a physician, a health technician, researcher, data scientist, or other individual involved in a sleep study or associated with a healthcare provider of the subject.

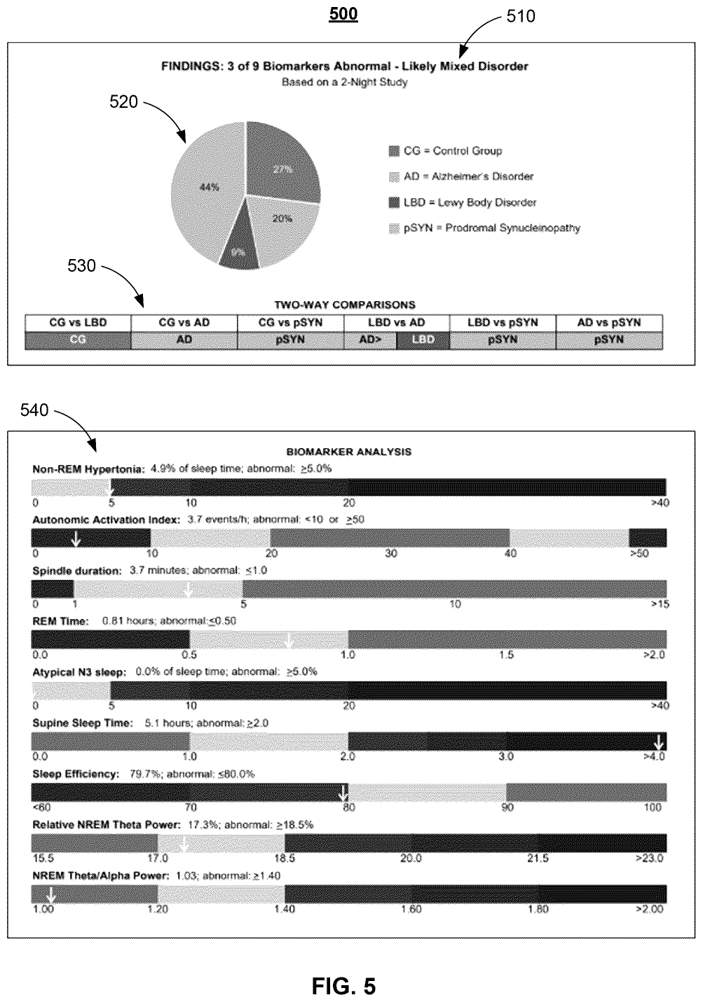

FIG. 5 illustrates a screen 500 of a graphical user interface, comprising a report of the risk severity, according to an embodiment. As illustrated, NDD risk and severity may be presented using both across-group and within-group classification results to assist with clinical interpretation. Screen 500 may comprise the overall risk severity 510, coupled with a chart 520 (e.g., pie chart) depicting the distribution of risk probabilities, output by the classifier in subprocess 330, for the normal class and each of the NDD class(es).

Screen 500 may also comprise a two-way comparison 530 of each pair of classes, with the winner of each comparison determined based on the class with at least a 60% risk probability. If the pair of classes both have a risk probability between 40-60%, then both classes may be listed, with the class having the greater risk probability listed to the left, and the class having the lower risk probability listed to the right. Preferably, the winning class, rather than the actual risk probabilities, is listed in each two-way comparison 530, since two-way risk probabilities have distorted values (e.g., 99% vs. 1%), and the difference in absolute values may be confusing or a statistical illusion with respect to absolute severity.

Screen 500 may also comprise an analysis 540 of each of the sleep biomarker(s) that was input to the classifier in subprocess 330 or contributed to the overall classification and risk probabilities. For each of the sleep biomarker(s), analysis 540 may visually illustrate the normal and abnormal ranges of values for that sleep biomarker and the position of the value of that sleep biomarker, for the specific subject, along a spectrum that includes both of the normal and abnormal ranges. This assists the user in interpreting the subject's results, including identifying how abnormal (i.e., outside the normal range) or normal (i.e., within the normal range) the subject's value is for each sleep biomarker.

### 4. SLEEP BIOMARKERS

Examples of the sleep biomarker(s) will now be described in detail. It should be understood that each described sleep biomarker may be one of the sleep biomarker(s) that is derived, in subprocess 320, based on the physiological data. The sleep biomarker(s) may comprise or consist of a single one of the described sleep biomarkers, all of the described sleep biomarkers, any subset of the described sleep biomarkers, all or any subset of the described sleep biomarkers with additional sleep biomarkers that are not specifically described herein, or a completely different set of sleep biomarkers than are specifically described herein. In an embodiment, the sleep biomarker(s) comprises a measure of time spent in rapid eye movement (REM) sleep, a measure of spindle activity (e.g., spindle duration, spindle density, etc.), a measure of atypical slow-wave (N3) sleep, a measure of non-rapid eye movement (NREM) hypertonia (NRH), a measure of autonomic activation, relative theta-wave power, a ratio of theta-wave power to alpha-wave power, a measure of sleep efficiency, sleep duration in the supine position, a measure of electroencephalographic (EEG) slowing, a measure of rapid eye movement (REM) sleep without atonia (RSWA) events, a pattern of oscillatory events, and/or the like.

### 4.1. Rapid Eye Movement (REM)

EMG intrusions in the EEG signal of the physiological data may occur as the result of movement by the subject and/or RSWA-like excursions that result from underlying REM sleep behavioral disorder (RBD). Since EMG activity is greater during NREM sleep than during REM sleep, the incorrect staging of NREM sleep as REM sleep may result in elevated RSWA scores and false positives. In contrast, the incorrect staging of REM sleep as NREM sleep may result in false negatives. Thus, REM sleep may be automatically staged to address this confounding factor.

In an embodiment, the sleep biomarker(s) comprise a measure of REM sleep, based on this automated staging. REM sleep is typically measured as either a time duration spent in REM sleep or a percentage of total sleep duration spent in REM sleep. In an embodiment, the sleep biomarkers comprise the time duration spent in REM sleep, rather than the percentage of total sleep time spent in REM sleep, to avoid distortions in the metric caused by short sleep durations. Research indicates that both subjects in the progressive supranuclear palsy (PSP) group and subjects in the LBD group exhibit less REM sleep compared to subjects in the control, MCI, PD, and AD groups (P<0.002). In addition, subjects in the AD group had less REM sleep compared to the control group (P<0.0005).

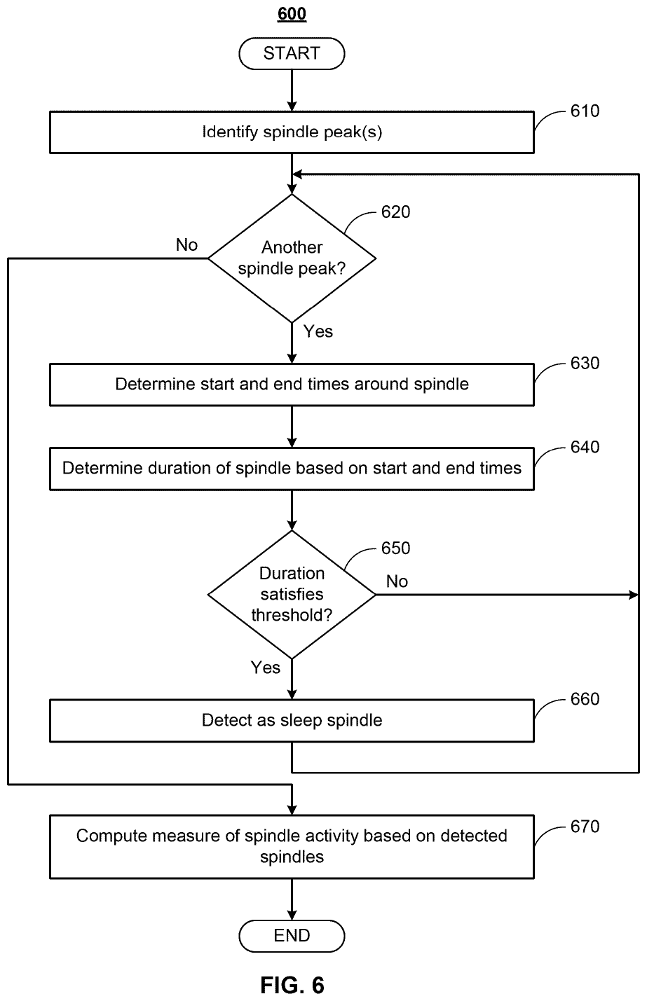

In an embodiment, the sleep biomarker(s) comprise a measure of spindle activity. FIG. 6 illustrates a process 600 for deriving the measure of spindle activity, according to an embodiment. Process 600 may be implemented by server application 112, client application 132, sleep-monitoring system 140, a combination of server application 112 and client application 132, a combination of sleep-monitoring system 140 and server application 112, a combination of sleep-monitoring system 140 and client application 132, or a combination of sleep-monitoring system 140, server application 112, and client application 132. While process 600 is illustrated with a certain arrangement and ordering of subprocesses, process 600 may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

### 4.2. Spindle Activity

Subprocess 610 may identify one or more spindle peaks. A spindle peak may be identified as comprising a burst in both the sigma-wave power and alpha-wave power, within the physiological data. The combination of sigma-wave and alpha-wave powers may ensure that both fast and slow sleep spindles are detected, irrespective of amplitude. In an embodiment, the beta-wave and EMG signals may also be considered in subprocess 610. For example, subprocess 610 may comprise computing the absolute power in the alpha-wave (i.e., 8-12 Hz), sigma-wave (i.e., 12-16 Hz), beta-wave (i.e., 18-28 Hz), and EMG (i.e., <40 Hz) signals, and the total power across all four bands (e.g., at a sampling rate of four samples per second), and identifying peaks in the sigma-wave power.

A spindle peak may be determined when one or more criteria are satisfied. A first criterion, representing dominant sigma-wave power, may be that absolute sigma-wave power is greater than or equal to 3.0, relative sigma-wave power is greater than 2.0, the difference between sigma-wave and alpha-wave powers is less than 2.0, the relative beta-wave power divided by the relative sigma-wave and alpha-wave powers is less than 0.25, and the relative EMG power is less than 7.5. A second additional or alternative criterion, representing mixed sigma-wave and alpha-wave power, may be that absolute sigma-wave power is greater than or equal to 2.25, the sum of absolute sigma-wave and alpha-wave powers is greater than 3.5, the difference between sigma-wave and alpha-wave power is less than 2.0, the sum of relative sigma-wave and relative alpha-wave powers is greater than 0.35, the relative beta-wave power divided by the relative sigma-wave and alpha-waver powers is less than 0.25, and the relative EMG power is less than 7.5. A third additional or alternative criterion, representing mixed sigma-wave and alpha-wave power, may be that absolute sigma-wave power is greater than or equal to 2.25, the sum of absolute sigma-wave and alpha-wave powers is greater than 4.0, the difference between sigma-wave and alpha-wave powers is less than 2.0, the sum of relative sigma-wave and relative alpha-wave power is greater than 0.30, the relative beta-wave power divided by the relative sigma-wave and alpha-wave powers is less than 0.25, and the relative EMG power is less than 7.5.

Subprocess 620 may determine whether or not another spindle peak, identified in subprocess 610, remains to be evaluated. When another spindle peak remains to be evaluated (i.e., “Yes” in subprocess 620), process 600 may select the next spindle peak and proceed to subprocess 630. Otherwise, when no more spindle peaks remain to be considered (i.e., “No” in subprocess 620), process 600 may proceed to subprocess 670.

Subprocess 630 may determine the start time and the end time around the spindle peak, selected in subprocess 620, based on one or more thresholds. In particular, a threshold may be applied to one or more of the signals (e.g., sigma-wave, alpha-wave, beta-wave, and/or EMG signals) on each side of the selected spindle peak. The time at which the signal(s), preceding the selected spindle peak, cross the threshold may be marked as the start time, and the time at which the signal(s), following the selected spindle peak, re-cross the threshold may be marked as the end time.

Subprocess 640 may determine the duration of the sleep spindle based on the start time and the end time, determined in subprocess 630. In particular, subprocess 640 may calculate the time duration between the start time and the end time (e.g., by subtracting the start time from the end time).

Subprocess 650 may determine whether or not the duration, determined in subprocess 640, satisfies a threshold. In particular, subprocess 650 may determine whether or not the duration is greater than or equal to a first threshold, such as 0.5 seconds. Alternatively or additionally, subprocess 650 may determine whether or not the duration is less than a second threshold, such as 3.0 seconds. Other thresholds may be applied to the signals to reduce the incidence of medication-related false positives. For example, the absolute sigma-wave power divided by the median absolute sigma-wave power may be required to be less than a threshold (e.g., 1.3), the beta-wave power may be required to be greater than or equal to a threshold (e.g., 0.25), the relative EMG power may be required to be less than a threshold (e.g., 10%), and/or the like. The use of these thresholds may filter out increases in alpha-wave power that are attributable to cortical arousals. When determining that the duration satisfies the threshold(s) (i.e., “Yes” in subprocess 650), process 600 may proceed to subprocess 660. Otherwise, when determining that the duration does not satisfy the threshold(s) (i.e., “No” in subprocess 650), process 600 may return to subprocess 620.

Subprocess 660 may detect the sleep spindle. In other words, subprocess 660 may detect the sleep spindle when the duration of the sleep spindle satisfies the threshold(s) (i.e., “Yes” in subprocess 650). In particular, subprocess 660 may add the sleep spindle to a set of detected sleep spindles that is persistently maintained throughout process 600.

Subprocess 670 may compute the measure of spindle activity, to be used as a sleep biomarker. The measure of spindle activity may comprise or consist of the duration of spindle activity. This spindle duration may be based on the duration of each detected sleep spindle. For instance, the spindle duration may be calculated as the sum of the durations of all detected sleep spindles during NREM or stage-two sleep (N2) and slow-wave or stage-three (N3) sleep. Alternatively, the measure of spindle activity may comprise or consist of the spindle density. The spindle density may be calculated as the number of detected sleep spindles during N2 and N3 sleep divided by the total time duration of N2 and N3 sleep. It should be understood that there are other ways to quantify spindle activity, including differentiating between fast and slow spindle activity, localizing the source of spindle activity, tallying spindle activity, and/or the like. Any manner of quantifying spindle activity is contemplated by the measure of spindle activity computed in subprocess 670.

Research indicates that spindle duration is reduced in both the PSP and LBD groups compared to control, iRBD, MCI, and ADem groups (P<0.025). A threshold for abnormal spindle duration set at one minute or less yields a rater operating characteristic (ROC) area under the curve (AUC) of 0.65, with sensitivity of 0.66 and specificity of 0.64 when ADem, LBD/DLB and PSP groups were compared to the control, iRBD, MCI, and PD groups.

### 4.3. Atypical Slow-Wave (AN3) Sleep

In an embodiment, the sleep biomarker(s) comprise a measure of atypical slow-wave (AN3) sleep. AN3 sleep may be detected using techniques that recognize suppressed theta-wave power, relative to delta-wave power, and low sigma-wave power, relative to alpha-wave power.

When analyses were conducted with AN3 sleep measured as a percentage of total sleep duration, the LBD group exhibited significantly greater AN3 sleep as compared to the AD and PD groups (both p<0.02), and the MCI, iRBD, and control groups (all p<0.005). When using an abnormal AN3 cut-off of greater than or equal to 4%, the proportion of the LBD group with abnormal AN3 sleep (42.1%) was significantly greater than the MCI (14.6%), iRBD (5.3%) and control (6.6%) groups (all p<0.05).

### 4.4. Non-Rapid Eye Movement (NREM) Hypertonia (NRH)

In an embodiment, the sleep biomarker(s) comprises a measure of NREM hypertonia. Generally, an episode of NREM hypertonia is identified as power in the EEG signal being persistently elevated by greater than 40 Hz, relative to the delta-wave, theta-wave, and sigma-wave powers, within each epoch (e.g., 30-second time interval). In an embodiment, variability thresholds may be applied within and across contiguous epochs, to ensure that EMG bursts, attributable to sleep-disorder breathing arousals, movements, or the like, are not mischaracterized as NREM hypertonia. Rules may also be applied to combine epochs into contiguous NRH blocks. In addition, NRH blocks within a time duration immediately following sleep onset (e.g., the first ten minutes of sleep) and/or or within a time duration of an upright position (e.g., ten minutes of upright time) may be excluded.

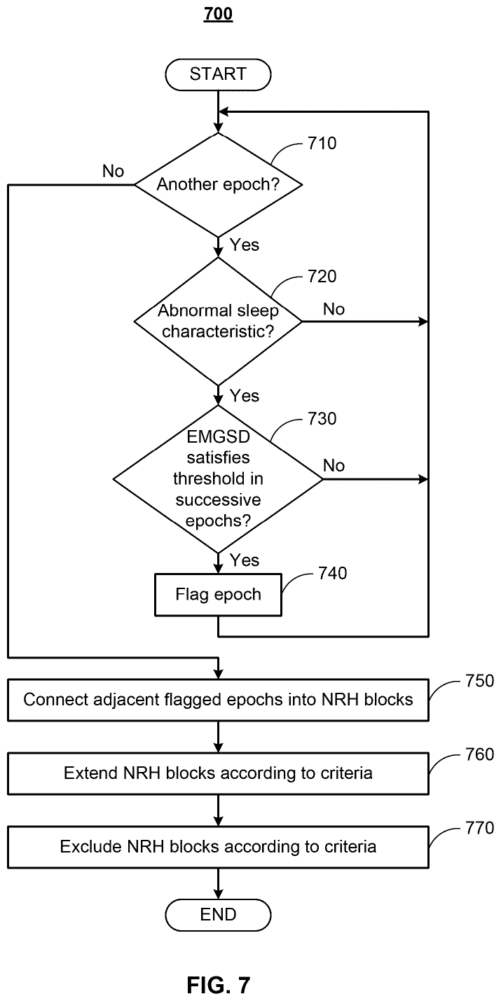

FIG. 7 illustrates a process 700 for deriving episode(s) of NREM hypertonia, according to an embodiment. Process 700 may be implemented by server application 112, client application 132, sleep-monitoring system 140, a combination of server application 112 and client application 132, a combination of sleep-monitoring system 140 and server application 112, a combination of sleep-monitoring system 140 and client application 132, or a combination of sleep-monitoring system 140, server application 112, and client application 132. While process 700 is illustrated with a certain arrangement and ordering of subprocesses, process 700 may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

The physiological data may be divided into a plurality of epochs. Each epoch is distinct from every other epoch and has the same fixed time duration. The time duration of an epoch may any suitable value. In an embodiment, each epoch has a time duration of thirty seconds. In this case, it should be understood that there may be hundreds of epochs in the physiological data (e.g., almost one thousand for eight hours of sleep). For each of the plurality of epochs, represented in the physiological data, process 700 may iterate through subprocess 710, 720, 730, and/or 740.

Subprocess 710 may determine whether or not another epoch, from among the plurality of epochs, remains to be evaluated. When another epoch remains to be evaluated (i.e., “Yes” in subprocess 710), process 700 may select the next epoch and proceed to subprocess 720. Otherwise, when no more epochs remain to be evaluated (i.e., “No” in subprocess 710), process 700 may proceed to subprocess 750.

Subprocess 720 may determine whether or not the epoch, currently under evaluation, exhibits an abnormal sleep characteristic based on one or more signals in the physiological data. These signal(s) may represent delta-wave power without ocular activity, theta-wave power, sigma-wave power, electromyographic (EMG) power, and/or the like. When determining that the epoch exhibits the abnormal sleep characteristic (i.e., “Yes” in subprocess 720), process 700 may proceed to subprocess 730. Otherwise, when determining that the epoch does not exhibit the abnormal sleep characteristic (i.e., “No” in subprocess 720), process 700 may return to subprocess 710.

In an embodiment, subprocess 720 may comprise applying a set of rules to determine whether or not the epoch exhibits an abnormal sleep characteristics. For example, subprocess 720 may determine that the epoch does not exhibit the abnormal sleep characteristic when the delta-wave power without ocular activity exceeds a first threshold (e.g., 17). This rule rejects epochs with a high probability of representing wakefulness of the subject. Subprocess 720 may also identify whether or not the epoch has elevated EMG power with limited cross-epoch EMG variability (e.g., EMG power greater than 0.43, standard deviation of EMG (EMG-SD) power across successive epochs less than 0.33, and the ratio of EMG-SD power to EMG power less than 0.375), and determine that the epoch does not exhibit the abnormal sleep characteristic when the epoch does not have this elevated EMG power with limited cross-epoch EMG variability.

In an embodiment, the rules are designed to detect elevated, but steady EMG power in the EEG signal. The relationships between delta-wave, theta-wave, and EMG powers may then be tested using two regression equations, developed with five epochs selected across a range of EEG amplitudes representing NREM hypertonia. One regression equation may utilize the actual ratio of theta-wave power to EMG power to set the minimum threshold for delta-wave power to EMG power, and the other regression equation may utilize the actual ratio of delta-wave power to EMG power to set the minimum threshold for the ratio of theta-wave power to EMG power. An epoch may be determined to represent NREM hypertonia when both of these minimum thresholds are satisfied.

In addition, the ratio of sigma-wave power to EMG power may be used to limit epochs with a normal sleep characteristic from being recognized as NREM hypertonia. In particular, subprocess 720 may calculate a delta threshold based on a theta-EMG ratio of the theta-wave power and the EMG power (e.g., delta threshold=(1.207*theta-wave power/EMG power)²−(3.6949*theta-wave power/EMG power)+5.6299), calculate a theta threshold based on a delta-EMG ratio of the delta-wave power without ocular activity and the EMG power (e.g., theta threshold=(1.669*natural-log(delta-wave power without ocular activity/EMG power))+0.1687), determine whether or not the delta-EMG ratio is within a first range based on the delta threshold (e.g., the delta-EMG ratio is greater than or equal to 2.75, the delta-EMG ratio is greater than the delta threshold, and the delta threshold is less than 15), determine whether or not the theta-EMG ratio is within a second range based on the theta threshold (e.g., the theta-EMG ratio is less than the theta threshold), determine whether or not a sigma-EMG ratio of the sigma-wave power and the EMG power is within a third range (e.g., the sigma-EMG ratio is less than or equal to 1.20), determine that the epoch does not exhibit the abnormal sleep characteristic when either the delta-EMG ratio is not within the first range, the theta-EMG ratio is not within the second range, or the sigma-EMG ratio is not within the third rage, and determine that the epoch exhibits the abnormal sleep characteristic when the delta-EMG ratio is within the first range, the theta-EMG ratio is within the second range, and the sigma-EMG ratio is within the third range.

Subprocess 730 may determine whether or not the standard deviation of the EMG power (EMGSD) satisfies a predefined threshold within a set of two or more epochs that includes the epoch currently being evaluated. The standard deviation of the EMG power may satisfy the predefined threshold when it is less than the predefined threshold (e.g., 0.15). The two or more epochs may be successive epochs that include the epoch under evaluation, and may consist of two epochs, three epochs, or more epochs. In the case of three or more epochs, the set of epochs may comprise or consist of the epoch that immediately precedes the epoch under evaluation, the epoch under evaluation, and the epoch that immediately follows the epoch under evaluation. As an alternative or additional example, the set of epochs may comprise or consist of the two epochs immediately preceding the epoch under evaluation, and the epoch under evaluation. As yet another alternative or additional example, the set of epochs may comprise or consist of the epoch under evaluation, and the two epochs immediately following the epoch under evaluation. When determining that the standard deviation of the EMG power satisfies the predefined threshold (i.e., “Yes” in subprocess 730), process 700 may proceed to subprocess 740. Otherwise, when determining that the standard deviation of the EMG power does not satisfy the predefined threshold (i.e., “No” in subprocess 730), process 700 may return to subprocess 710.

Subprocess 740 may flag any epoch that is determined to exhibit the abnormal sleep characteristic, as determined in subprocess 720, and for which the standard deviation of the EMG power satisfies the predefined threshold in successive epochs, as determined in subprocess 730. It should be understood that a flagged epoch represents an epoch that reflects an episode of NREM hypertonia.

Subprocess 750 may connect two or more of the plurality of epochs, that each exhibits the abnormal sleep characteristic and for which the standard deviation of the EMG power satisfies the predefined threshold within the set of two or more epochs that includes the epoch under evaluation, into an NRH block. The connection of epochs may be in accordance with one or more rules and based on whether or not adjacent or nearby epochs have been flagged in subprocess 740. For example, subprocess 750 may analyze each flagged epoch, one at a time, and classify each flagged epoch as probable NRH when either at least one of the two immediately preceding epochs is also flagged, at least one of the two immediately following epochs is also flagged, and/or the immediately preceding and immediately following epochs are also flagged. Then, NRH blocks may be formed to span consecutive epochs based on those epochs that have been classified as probable NRH. For example, any four consecutive epochs classified as probable NRH may be connected into an NRH block, any six consecutive epochs in which four epochs have been classified as probable NRH and there are not two consecutive epochs that have not been classified as probable NRH may be connected into an NRH block, and/or any seven consecutive epochs in which five epochs have been classified as probable NRH and there are not two consecutive epochs that have not been classified as probable NRH may be connected into an NRH block. It should be understood that these are just examples, and that a different set of rules may be used to combine flagged epochs into NRH blocks. Subprocess 750 ensures that elevated EMG power is not associated with arousals that caused short-duration bursts of EMG activity (e.g., sleep-disordered breathing, dream enactment, periodic limb movement, etc.).

Subprocess 760 may extend one or more of the NRH blocks, formed in subprocess 750, to include one or more surrounding epochs, according to one or more criteria. For example, two NRH blocks may be combined when there are no more than two epochs between them. As another example, an NRH block comprising more than ten epochs may be extended to include any one or more flagged epochs that are separated from the NRH block by only one unflagged epoch. In this case, it should be understood that the unflagged epoch would also become part of the NRH block. As another example, an NRH block comprising more than eight epochs may be extended to include any two or more flagged epochs that are separated from the NRH block by only two unflagged epochs. In this case, it should be understood that the two unflagged epochs would also become part of the NRH block. Subprocess 760 may iteratively extend NRH blocks in both directions, in this manner, until no more extensions are possible.

Subprocess 770 may exclude any NRH block that satisfies one or more exclusion criteria. The exclusion criteria may comprise one or more of a presence of the NRH block within a predefined time duration (e.g., ten minutes) immediately following sleep onset, and/or the presence of the NRH block within a predefined time duration (e.g., ten minutes) of uprightness (i.e., when the physiological data indicate that the subject is upright for the predefined time duration). These exclusion criteria may be used since EMG is generally elevated after extended periods of wakefulness. Additionally or alternatively, the exclusion criteria may comprise the NRH block corresponding in time to a skin-electrode impedance greater than a predefined threshold impedance (e.g., 100 kΩ). This exclusion criterion limits the possibility of the derivation of NREM hypertonia being influenced by artifacts caused by periods with elevated skin-electrode impedance.

It should be understood that NRH blocks that are excluded by subprocess 770 are not included in the episodes of NREM hypertonia. In other words, the episode(s) of NREM hypertonia that are derived by process 700 consists of the NRH blocks that were produced by subprocess 710-760 and not excluded by subprocess 770. Each NRH block in this final set of NRH block(s) represents one NRH episode. Abnormal NREM hypertonia may be defined as a measure of the NRH episodes (e.g., number of NRH episodes, total duration of NRH episodes, percentage of duration of NRH episodes to total sleep time, etc.) satisfying (e.g., being greater than or equal to) a predefined threshold.

Research indicates that abnormal NREM hypertonia was significantly associated with subjects in the LBD, PSP, PD and iRBD groups, as compared to subjects in the AD, MCI, and control groups (P<0.0001). A threshold for abnormal NRH of greater than or equal to 5% of sleep time yielded a rater operating characteristic area under the curve of 0.77, with a sensitivity of 0.75 across the iRBD, PD, PDD, DLB and PSP groups, and a specificity of 0.79 for the control, MCI, and ADem groups. The frequency of abnormal NREM hypertonia in the PSP group (92%) was significantly greater than the MCI (26%), AD (17%), and NC (16%) groups (all P<0.0001), and the PD group (56%) (P<0.05), but not the DLB/PDD (81%) and iRBD (74%) groups. In an embodiment, the severities of NREM hypertonia and RSWA events are compared to each other, in order to rate the progression of the disease and/or as a measure of likelihood of phenoconversion to dementia.

### 4.5. Autonomic Activation

In an embodiment, the sleep biomarker(s) comprise a measure of autonomic activation. Autonomic dysfunction, which may be detected by quantifying changes in heart rate during sleep, may occur in patients with synucleinopathy-related disorders, severe sleep-disordered breathing, or periodic limb movements. Heart-rate variability is a conventional measure of autonomic dysfunction, that may be estimated through a Fast-Fourier transform or other means, to precisely identify heart-rate frequency. Alternatively, heart-rate variability may be measured using an autonomic activation index (AAI). In this case, thresholds are selected to identify brief increases and/or decreases in heart rate, relative to previous or subsequent temporally defined time windows of heart-rate activity. These events may then be tallied and indexed according to sleep time.

In an embodiment, heart-rate variability is measured using a six beat-per-minute (bpm) increase and/or decrease, compared to the previous and subsequent ten seconds. One skilled in the art may apply different heart rate increases/decreases and time-windowed comparisons to detect these autonomic activation events and obtain an autonomic activation index based on the number of events per hour of overall non-REM and/or REM sleep.

In an embodiment, the measure of autonomic activation (e.g., heart-rate variability or autonomic activation index) is measured selectively during non-REM sleep to assess abnormal parasympathetic activity and during REM sleep to assess abnormal sympathetic activity. In an alternative embodiment, the measure of autonomic activation is measured across all sleep stages to limit the missing data that might occur in subjects with limited REM sleep duration.

In a preferred embodiment, the extraction of the measure of autonomic activation only occurs when signal quality is acceptable for a sufficient period to proportionally represent the entire sleep duration. In one embodiment the measure of autonomic activation is excluded from analysis if the ECG or pulse-rate signals are of inadequate quality for more than 30% of the sleep duration. When the autonomic activation index is used as a continuous measure, it may recognize abnormally low values as autonomic dysfunction and abnormally high values as a sleep disorder (e.g., obstructive sleep apnea, periodic limb movements, etc.).

Research found that AAI values decreased in DLB/PDD groups compared to the AD and MCI groups (both p<0.03) and control group (p<0.0005), while the AAI values for the PD group were less than the AD and MCI groups (both <0.05) and the control group (p<0.002). The proportion of cases with abnormal AAI values (<10 events/hour) was greatest in the LBD group, and greater than in the AD (p<0.02), MCI (p<0.02) and control (p<0.0001) groups, while AAI values in the PD and iRBD groups were greater than in the control group (p<0.007 and p<0.05, respectively). An abnormal autonomic activation index also delivered a specificity of 0.87 for the control group achieved with rater operating characteristic areas under the curve and sensitivities ranging from 0.75 and 0.63, respectively, for the control group vs. LBD group, with slightly lower AAI values achieved when LBD and PD groups are combined or additionally joined by the prodromal synucleinopathy iRBD group.

### 4.6. Sleep Efficiency

In an embodiment, the sleep biomarker(s) comprise a measure of sleep efficiency. Both the duration of sleep, as well as the amount of sleep one obtains while attempting to sleep (i.e., sleep efficiency), are important when considering neurodegenerative disorders. Research found that the control group had greater sleep duration compared to the iRBD and PSP groups (P<0.025), while the sleep efficiencies of the PSP group were less than the LBD, PD, iRBD, AD, MCI, and control groups (P<0.005). The sleep efficiencies of the LBD group were less than the control and MCI groups (P<0.002), and the sleep efficiencies of the AD group were less than the control group (P<0.0005).

### 4.7. Supine Sleep

In an embodiment, the sleep biomarker(s) comprise sleep duration in a supine position. Sleep position reportedly influences the efficiency of glymphatic clearance of beta amyloid, with the lateral position (i.e., on the subject's side) being more efficient than the supine position (i.e., on the subject's back). The lateral and supine positions are both superior to the typical head position of humans during wakefulness. Assuming that the improvement in glymphatic clearance results from the partial collapse of the internal jugular veins above the heart, when lying laterally, then sleeping position should preferably be measured on the neck or head, to better establish the relationship of the left and right internal jugular veins relative to the heart. Such an embodiment would optimize the measurement of the sleep position versus glymphatic clearance relationship.

Research found that sleep duration in the supine position was lower in the control group compared to the LBD and PD, AD, and MCI groups (all p<0.002). Using the threshold of greater than or equal to two hours, the proportion of subjects with abnormal time in the supine position increased from the control group (39%) to the LBD and PD (71%), AD (69%), and MCI (59%) groups (all p<0.025). When compared to the control group, the odds of having a neurodegenerative disorder were 3.6 for the LBD and PD group, 3.5 for the AD group, and 2.2 for the MCI group.

### 4.8. Electroencephalographic (EEG) Slowing

In an embodiment, the sleep biomarker(s) comprise a measure of electroencephalographic (EEG) slowing. EEG slowing is common to all dementias, potentially because of atrophy, and may be observed as a combination of decreased delta-wave activity and increased theta-wave activity. In an embodiment, theta-wave power is measured relative to other EEG frequencies in order to identify slowing related to cognitive decline.

Research found significant EEG slowing in both the MCI and AD groups, when compared to a control group based on increased ratios of theta waves to alpha waves across all sleep stages (P<0.001). An alternative measure of EEG slowing, attributed to cognitive decline, is based on the ratio between theta-wave power and the total EEG power (e.g., from 0.1 to 128 Hz). In an embodiment, the total power is restricted to the delta-wave, theta-wave, alpha-wave, sigma-wave, and beta-wave frequencies, in order to minimize the influence of muscle or movement artifacts. One skilled in the art will recognize that various combinations of frequency bands may be used to identify excessive EEG slowing.

4.9. REM Sleep without Atonia (RSWA)

In an embodiment, the sleep biomarker(s) comprise a measure of rapid eye movement (REM) sleep without atonia (RSWA) events. Visual scoring rules for phasic and tonic RSWA events are variably defined depending on the clinical or research application. A commonly used definition of a phasic RSWA event is a burst in EMG activity of one-hundred milliseconds or greater and at least four times greater than the lowest REM baseline EMG activity. A commonly used definition of a tonic RSWA event is EMG activity that is elevated to at least two times greater than the REM baseline EMG activity for fifteen seconds or more. Visually scored RSWA events may also consider the quality of the EMG signal, the influence of ECG artifacts, and the timing of the EMG burst relative to sleep-disordered breathing and/or snoring. RSWA events may be tallied by first separating each of the plurality of epochs (e.g., thirty seconds in duration) in the physiological data into ten mini-epochs (e.g., three seconds in duration), and then counting the number of mini-epochs that are encroached by either a phasic or a tonic RSWA event, as defined above. It should be understood that if the physiological data comprise one-thousand epochs, there will be ten-thousand mini-epochs in this exemplary embodiment.

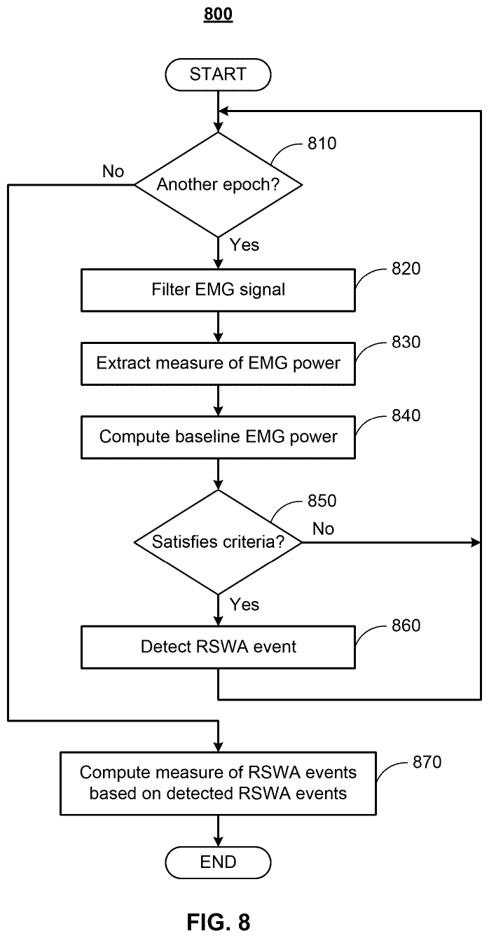

FIG. 8 illustrates a process 800 for deriving a measure of RSWA events, according to an embodiment. Advantageously, process 800 enables the detection of RSWA events to be automated, such that visual detection is no longer required and human error can be eliminated. Process 800 may be implemented by server application 112, client application 132, sleep-monitoring system 140, a combination of server application 112 and client application 132, a combination of sleep-monitoring system 140 and server application 112, a combination of sleep-monitoring system 140 and client application 132, or a combination of sleep-monitoring system 140, server application 112, and client application 132. While process 800 is illustrated with a certain arrangement and ordering of subprocesses, process 800 may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

RSWA events may be detected by evaluating each of the plurality of epochs represented in the physiological data. In particular, for each of the plurality of epochs, process 800 may iterate through subprocess 810, 820, 830, 840, 850, and/or 860.

Subprocess 810 may determine whether or not another epoch, from among the plurality of epochs, remains to be evaluated. When another epoch remains to be evaluated (i.e., “Yes” in subprocess 810), process 800 may select the next epoch and proceed to subprocess 820. Otherwise, when no more epochs remain to be evaluated (i.e., “No” in subprocess 810), process 800 may proceed to subprocess 870.

Subprocess 820 may filter the EMG signal in the physiological data for the epoch under evaluation. The filtering may comprise applying one filter or a plurality of different filters. For example, EMG signals that were acquired from the head and/or limbs of the subject may be filtered to remove ECG artifacts (i.e., heartbeat artifacts), in order to avoid RSWA misclassifications.

Subprocess 830 may extract a measure of EMG power from the EMG signal that was filtered in subprocess 820. By definition, an RSWA event occurs during REM sleep with atypical muscle activity. Conventionally, the automated staging of REM sleep assumes atonia. Thus, subprocess 830 may stage the REM sleep while accounting for muscle movements and/or movements that result from RBD-related dream enactment. Subprocess 830 may also assess the signal quality of the EMG signal to identify and reject periods of low EMG signal quality, in order to limit misclassifications attributable to increased environmental artifacts or other artifacts that cannot be readily removed by filtering in subprocess 820. Subprocess 830 may then compute the continuum of EMG power (i.e., greater than 40 Hz) across the epoch under evaluation. The EMG power may be calculated at a resolution of sixteen times per second. In addition, the integration/envelope transform (e.g., root-mean-square function) may be applied to the EMG signal to quantify bursts and/or elevated EMG activity.

Subprocess 840 may compute a baseline EMG power during REM sleep for the epoch under evaluation. EMG activity during REM sleep in which the EMG signal is distorted due to environmental noise (e.g., introduced as a result of partially affixed sensors or during gross movements, etc.) may be excluded from analysis. The baseline EMG power may be computed based on the mean values of EMG power during a contiguous set of epochs, including the epoch under evaluation (e.g., including at least one epoch preceding and/or following the epoch under evaluation). Alternatively, the mean values of EMG power may be computed during the same three epochs after exclusion of periods during loud snoring or gross movement. In yet another alternative embodiment, the baseline EMG power is computed from the average of EMG power across all non-excluded epochs representing REM sleep.

Subprocess 850 may determine whether or not the measure of EMG power, extracted in subprocess 830, satisfies one or more criteria, representing excursions in the EMG signal. The criteria may utilize the baseline EMG power, computed in subprocess 840. For example, the criteria may comprise a ratio of the EMG power to the baseline EMG power satisfying (e.g., being greater than or equal to) a predefined threshold. Additionally, the criteria may require the EMG power to be greater than a minimum and/or less than a maximum value when the predefined threshold is satisfied. For instance, a phasic RSWA event may be detected when the ratio of EMG power to baseline EMG power is greater than or equal to two and the EMG power is greater than or equal to 2.0, and a tonic RSWA event may be detected when the ratio of EMG power to baseline EMG power exceeds a predefined threshold for the preponderance of a fixed time (e.g., fifteen-second) window. In an alternative embodiment, phasic and tonic RSWA events are detected by determining when the integrated power exceeds empirically derived amplitude and duration thresholds. In this case, a phasic RSWA event may be detected when the EMG power is greater than or equal to 2.0 for a minimum time duration (e.g., 250 milliseconds), and a tonic RSWA event may be detected when the mean integrated EMG power is two times greater than the baseline EMG power for a predefined number (e.g., five) of mini-epochs. When determining that the measure of EMG power satisfies the one or more criteria (i.e., “Yes” in subprocess 850), process 800 may proceed to subprocess 860. Otherwise, when determining that the measure of EMG power does not satisfy the one or more criteria (i.e., “No” in subprocess 850), process 800 may return to subprocess 810.

Both the EMG power and integrated EMG signal powers may be used or combined with multiple threshold layers that optimize the sensitivity and specificity of phasic and tonic RSWA event detection from any muscle or muscle combinations. In an embodiment, the thresholds are adapted to limit misclassification of snoring-related EMG activity as RSWA events. Another enhancement to RSWA detection may include the detection of the temporal timing of events, such that events that are highly periodic (e.g., consistent with heart rate) may be rejected as artifacts.

Subprocess 860 may detect an RSWA event in the epoch under evaluation when the measure of EMG power satisfies the one or more criteria, as determined in subprocess 850. In particular, the detected RSWA event may be added to a set of RSWA events that is maintained throughout process 800. In an embodiment, automatically detected RSWA events may be visually inspected to add or remove RSWA events, which may further reduce false positives and false negatives.

Subprocess 870 may compute the measure of RSWA events based on the detected RSWA events, if any. In an embodiment, the measure of RSWA events comprises or consists of the frequency and duration of the detected RSWA events, relative to all REM periods. For example, the measure of RSWA events may comprise RSWA density, which may be calculated based on the number of mini-epochs (e.g., three-second intervals) with RSWA events, relative to all mini-epochs of REM sleep. Alternatively, RSWA density may be calculated uniquely for phasic and tonic events and for each EMG signal (e.g., chin, arm, leg, etc.), and/or tallied across all types of RSWA events by and across all EMG signals. In an embodiment, if a sufficient number or percentage (e.g., 50% or greater) of min-epochs in an epoch are identified as having an RSWA event, then the sleep stage may be changed from REM to REM with RSWA. Notably, the measure of RSWA events, as a sleep biomarker, is a good predictor for pSYN and LBD, and therefore, enhances the predictive ability of the classifier.

Conventionally, the diagnosis of RBD is made during laboratory polysomnography, where the detection of RSWA events is combined with the use of video recordings to confirm dream-enactment behavior. Accordingly, in an embodiment, the detection of epochs having REM with RSWA (e.g., in subprocess 850) may be coupled with a questionnaire (e.g., in the subject's health record) to identify dream-enactment behavior. In this embodiment, the results of the dream-enactment questionnaire may be coupled with and included in a report summarizing the RSWA activity (e.g., in subprocess 350). Preferably, thresholds are applied to the results of the dream-enactment questionnaire and the proportion of epochs with REM with RSWA events, to assist the clinician in diagnosing RBD. This enables subjects to be diagnosed for RBD in their home, without the need for instructive video recordings.

### 4.10. Oscillatory Events

In an embodiment, the sleep biomarker(s) comprise a pattern of oscillatory events. The spatial relationship and timing of slow waves and sleep spindles, during normal sleep-dependent memory processing, have been correlated with amyloid and tau proteins. Thus, these patterns of oscillatory events may be used to assess the integrity of neural circuits controlling sleep's memory replay and provide a sleep biomarker of AD. Detection of these oscillatory events may comprise detecting and characterizing slow waves, which may include identifying the points of initiation and termination (i.e., minimum values or troughs), total amplitude change at the peak of the slow wave (i.e., u V change from initiation/termination to the maximum value), and durations (i.e., time lapse between each slow wave peak and associated troughs). In an embodiment, sleep spindles and theta waves are additionally detected, and coupled in time to various previously described slow-wave measures. The presence or absence of time-coupled sleep spindles and theta waves may be used to identify normal or abnormal conditions.

### 5. CLASSIFIER

One of the most important aspects to consider when applying statistical models or artificial intelligence (AI) techniques to classification is the selection of the classes, especially when the goal is to both differentiate and measure the severity between neurodegenerative disorders. Thus, in an embodiment, the classifier is developed using patient cohorts with distinctive heterogenous patterns across the severity progression (e.g., AD and LBD) and then cross-validated using patient cohorts with multiple pathways for phenoconversion. In a preferred embodiment, controls with normal cognition and/or groups of patients with prodromal indicators of future conversion to a specific dementia (e.g., iRBD typically converts to Parkinson's or Lewy bodies dementia) are used to measure and/or model disease progression and severity. In a particular implementation, a four-class model was used for the classifier. The four classes consisted of normal, AD, LBD, and iRBD (since 95% of patients diagnosed with RBD eventually convert to a Lewy body disease). The selection of AI statistical tools used combinations of sleep biomarkers, including some of those described herein, to obtain the risk probabilities for each of the classes. The selection also included adjustments for the probabilities of occurrence for each group. For example, AD is more prevalent than LBD, and thus, the likelihood of occurrence of AD is greater based on a memory deficit.

A proportion of LBD and PD patients may be diagnosed with RBD (e.g., using process 800). This information may be used to augment the risk profiling of subjects. In an embodiment, the pSYN group may be replaced with RSWA and dream-enactment variables used to diagnose RBD. In an alternative embodiment, pSYN may be relabeled as RBD in reports, when RSWA events are detected.

The table below depicts the accuracy of an implementation of the classifier across the four classes that were used for model development, with summary statistics based on absolute and trending toward diagnostic agreement and disagreement. Across the model development groups, the diagnostic disagreements for the AD, LBD, and control groups were limited to 3-10%. The iRBD group had the greatest number of diagnostic disagreements based on 32% being classified probably normal and another 25% being classified normal with indications of pSYN.

| | Training Data | | | |
|---|---|---|---|---|
| | AD | LBD | iRBD | CG |
| Risk Severity | n = 29 | n = 23 | n = 19 | n = 61 |
| Probably normal | 6.9% | 4.3% | 31.6% | 67.2% |
| Likely normal | | 4.3% | | |
| Likely normal, indications AD | 13.8% | 4.3% | | 8.2% |
| Likely normal, indications pSYN | 3.4% | 4.3% | 26.3% | 8.2% |
| Likely normal, indications Mixed | 3.4% | | | 4.9% |
| Likely AD | 6.9% | | | 4.9% |
| Likely LBD | | | | |
| Likely pSYN | | | | 3.3% |
| Likely Mixed | | | | |
| Probably AD | 51.7% | 4.3% | | 1.6% |
| Probably LBD | 3.4% | 65.2% | 10.5% | 1.6% |
| Probably pSYN | | 4.3% | 26.3% | |
| Probably Mixed | 10.3% | 8.7% | 5.3% | |
| Diagnostic Agreement | 69.0% | 78.3% | 42.1% | 88.5% |
| Likely trends toward Agreement | 17.2% | 4.3% | 26.3% | 0.0% |
| Likely trends toward Disagreement | 3.4% | 8.7% | 0.0% | 8.2% |
| Diagnostic Disagreement | 10.3% | 8.7% | 31.6% | 3.3% |

The table below depicts cross-validation group data, unique from the model development groups, based on a new set of healthy controls and subjects with MCI and PD. The primary differences in classification results, between the model development and cross-validation control groups, are attributable to a 6% increase in probably AD or pSYN cases. The proportion of MCI and PD cases, classified with normal sleep, were similar and less than 20%, while 13% of the PD subjects were classified normal with indications of AD.

| | Cross Validation | | |
|---|---|---|---|
| | CG | MCI | PD |
| Risk Severity | n = 66 | n = 41 | n = 16 |
| Probably normal | 62.1% | 17.1% | 12.5% |
| Likely normal | | | |
| Likely normal, indications AD | 4.5% | 7.3% | 12.5% |
| Likely normal, indications pSYN | 12.1% | 7.3% | 18.8% |
| Likely normal, indications Mixed | | 7.3% | |
| Likely AD | 1.5% | 12.2% | |
| Likely LBD | 1.5% | 4.9% | |
| Likely pSYN | 7.6% | 2.4% | 6.3% |
| Likely Mixed | 1.5% | 4.9% | 18.8% |
| Probably AD | 6.1% | 31.7% | 6.3% |
| Probably LBD | | | 12.5% |
| Probably pSYN | 3.0% | | 12.5% |
| Probably Mixed | | 4.9% | |
| Diagnostic Agreement | 78.8% | 61.0% | 50.0% |
| Likely trends toward Agreement | 0.0% | 22.0% | 18.8% |
| Likely trends toward Disagreement | 12.1% | 0.0% | 12.5% |
| Diagnostic Disagreement | 9.1% | 17.1% | 18.8% |

The table below depicts the test-retest consistency in the classification of a control group of fifty subjects, with 72% having the same classification after 8-58 months, with 18% shifting between probably and likely normal, and 10% showing an increase in severity.

| | | Likely | Likely | Likely | | | | |
|---|---|---|---|---|---|---|---|---|
| Risk | Prob. | Normal | Normal | Normal | Likely | Likely | Prob. | Prob. |
| Severity | Normal | (AD) | (pSYN) | (Mixed) | AD | pSYN | AD | LBD |
| Prob. | 52.0% | | 4.0% | 2.0% | | | | |
| Normal | | | | | | | | |
| Likely | 4.0% | | | 2.0% | | | | |
| Normal | | | | | | | | |
| (AD) | | | | | | | | |
| Likely | 6.0% | 2.0% | 6.0% | | | 2.0% | | |
| Normal | | | | | | | | |
| (pSYN) | | | | | | | | |
| Likely | 2.0% | | | | | 2.0% | | |
| pSYN | | | | | | | | |
| Likely | | | | | 2.0% | | | |
| Mixed | | | | | | | | |
| Prob. | | 6.0% | | | | | 4.0% | |
| AD | | | | | | | | |
| Prob. | | | | | | 2.0% | | 2.0% |
| LBD | | | | | | | | |
| Prob. | | | | | | 2.0% | | |
| pSYN | | | | | | | | |

The table below depicts the six-month test-retest classification consistency (i.e., longitudinal reliability of NDD risk severities) in the records of ten subjects in the LBD group. The consistency score of 90% included one subject who was classified with normal sleep at both times, and a second subject whose sleep was consistently classified as probably AD. A third subject's sleep was classified as probably LBD at baseline, but retested with mixed AD/LBD sleep patterns. 10.0% of subjects showed a shift in severity.

| | Prob. | Prob. | Prob. | Prob. |
|---|---|---|---|---|
| Risk Severity | Normal | LBD | Mixed | AD |
| Prob. Normal | 10.0% | | | |
| Likely AD/LBD Mixed | | 10.0% | | |
| Prob. LBD | | 50.0% | 10.0% | |
| Prob. AD | | | | 10.0% |
| Prob. Mixed | | | 10.0% | |

The table below depicts the six-month test-retest classification consistency (i.e., longitudinal reliability of NDD risk severities) in the records of nine subjects in the AD group. The consistency was not as strong as the LBD group. The overall test-retest agreement was 78%, and included one subject with sleep patterns that were consistently normal. The sleep patterns of another subject shifted from probably AD to likely AD, and a second subject's sleep was initially classified as normal with indications of AD, but demonstrated sleep consistent with LBD upon retest. 22.2% of subjects showed a shift in severity.

| | Prob. | Likely Normal | Prob. | Prob. |
|---|---|---|---|---|
| Risk Severity | Normal | (AD) | AD | Mixed |
| Prob. Normal | 11.1% | | | |
| Likely AD | | | 11.1% | |
| Prob. Mixed | | | | 11.1% |
| Prob. AD | | | 55.6% | |
| Prob. LBD | | 11.1% | | |

### 6. EXAMPLE SLEEP SIGNALS

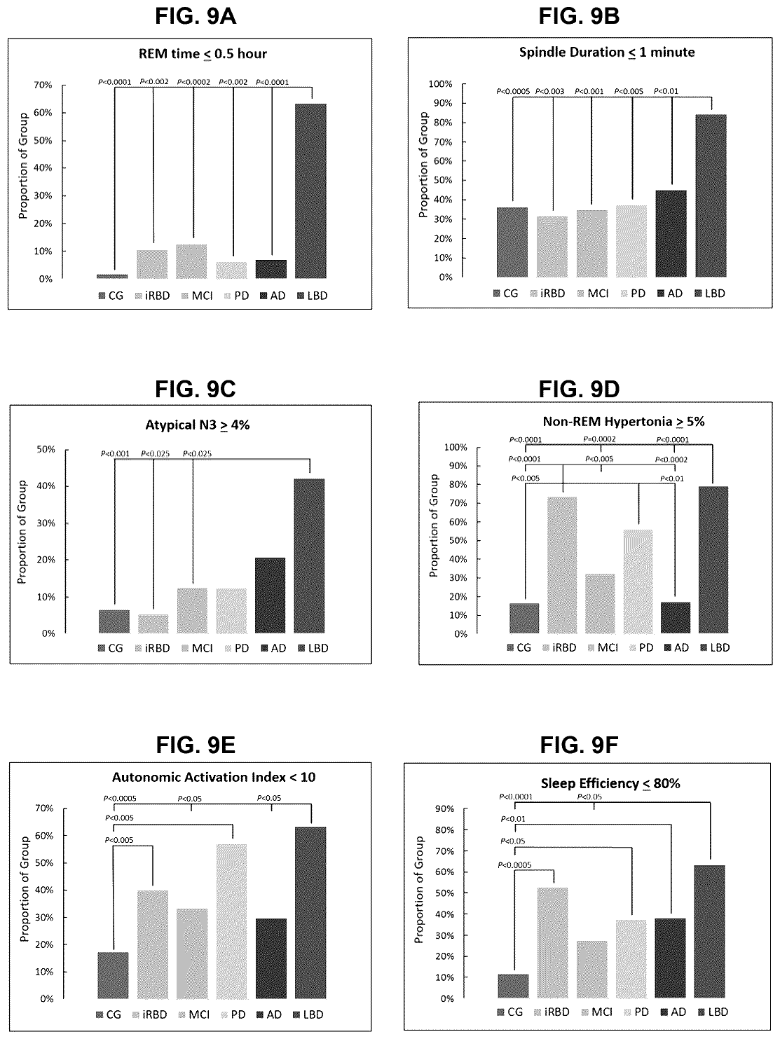

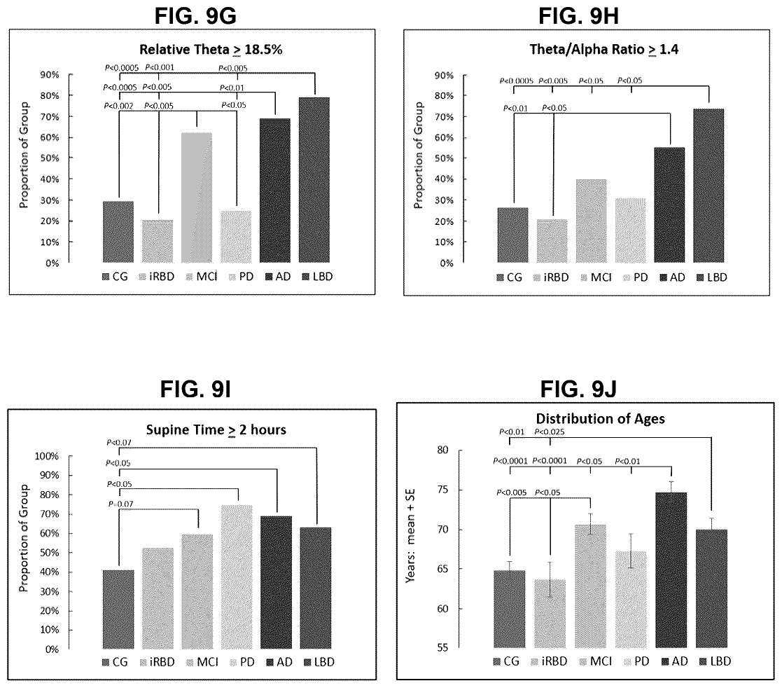

FIGS. 9A-9J illustrate examples of the proportion of subjects, classified into each neurodegenerative disorder, identified by individual sleep biomarkers, as well as the distribution of ages of the subjects, according to an embodiment. In this example, the classes included a normal class and five NDD classes (i.e., iRBD, MCI, PD, AD, and LBD). In addition, the plurality of sleep biomarkers comprised duration of REM sleep, overall spindle duration, percentage of sleep duration spent in AN3 sleep, percentage of sleep duration exhibiting NREM hypertonia, autonomic activation index, sleep efficiency, relative theta-wave power, the ratio of theta-wave power to alpha-wave power, and duration of sleep spent in the supine position.

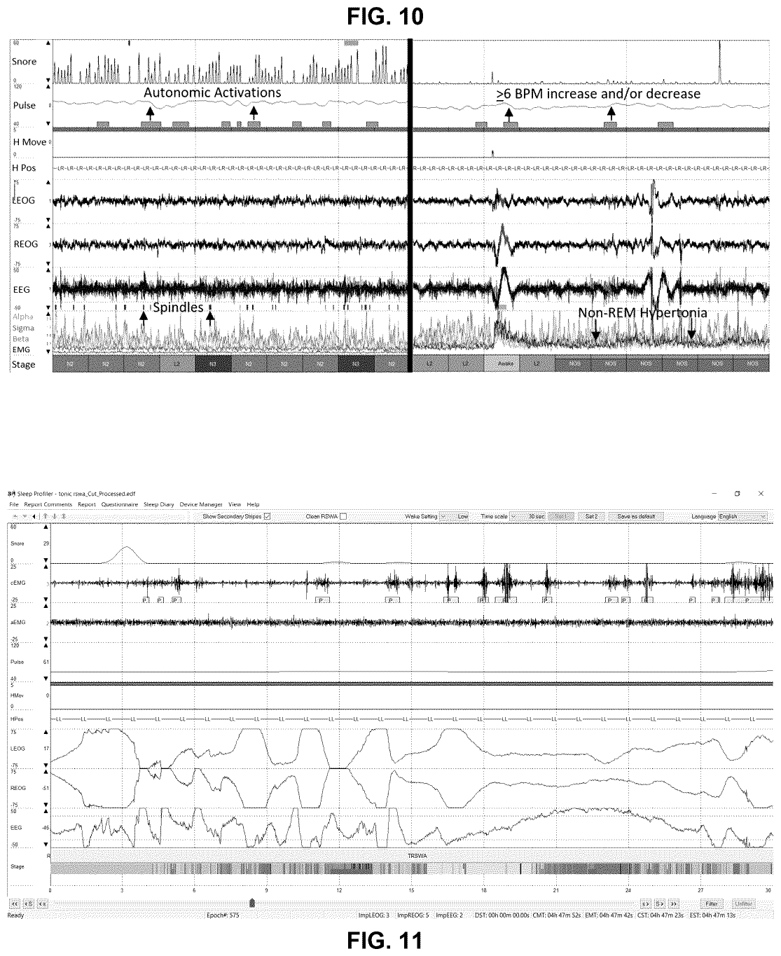

FIG. 10 illustrates an example set of sleep signals that may be used to derive a plurality of sleep biomarkers, according to an embodiment. The sleep signals demonstrate that increases and decreases in pulse rate may contribute to the autonomic activation index. The pulse signal, rather than the ECG signal, may be used to measure the heart-rate variability. In addition, it is shown that alpha-wave and sigma-wave power can be used to detect sleep spindles, and abrupt but steady elevations in EMG power can be used to detect NREM hypertonia.

FIG. 11 illustrates an example set of sleep signals that may be used to characterize REM sleep without atonia (RSWA), according to an embodiment. A single thirty-second epoch with automatically staged REM sleep is depicted. In the illustrated example, phasic RSWA events were detected, in the EMG signal acquired from the subject's chin, in eight of ten of the three-second mini-epochs. No RSWA events were detected in the EMG signal acquired from the subject's arm.

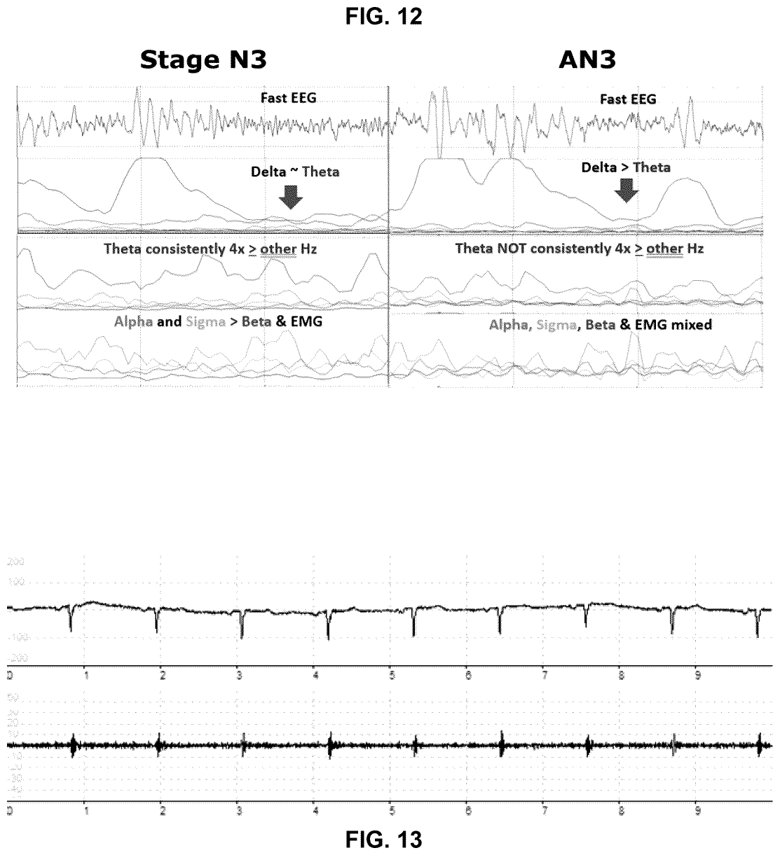

FIG. 12 illustrates an example set of sleep signals that highlights the signal patterns that are unique to the slow waves that appear during N3 sleep (on the left) versus those that appear during AN3 sleep (on the right), according to an embodiment. Two fifteen-second excerpts of the EEG signal are displayed, along with the associated power for the delta waves versus theta waves, and between alpha-wave, sigma-wave, beta-wave, and EMG powers. AN3 sleep (also known as sepsis-associated encephalopathy or polymorphic delta activity) exhibits suppressed theta-wave power relative to delta-wave power, and low sigma-wave power relative to alpha-wave power.

FIG. 13 illustrates an example EMG signal, before filtering (top) and after filtering (bottom), according to an embodiment. It can be seen that heartbeat artifacts in the EMG signal appear as EMG bursts. These EMG bursts could be misclassified as RSWA events after high-pass filtering, absent the disclosed embodiments. In particular, these EMG bursts may be removed in subprocess 820 of process 800.

### 7. REFERENCES

All of the following references are hereby incorporated herein by reference as if set forth in full:
Ref1: World Health Organization—Factsheet (www.who.int/en/news-room/fact-sheets/detail/dementia, accessed Feb. 21, 2021);
Ref2: Gooch C L, Pracht E, Borenstein A R. The burden of neurological disease in the United States: a summary report and call to action. Annals Neurol. 91 (2017) 479-484;
Ref3: Parnetti L, Lanari A, Silvestrelli G, et al. Diagnosing prodromal Alzheimer's disease: role of CSF biochemical markers. Mech Ageing Dev. 127 (2006) 129-132;
Ref4: Hampel H, Teipel S J, Fuchsberger T, et al. Value of CSF beta-amyloid1-42 and tau as predictors of Alzheimer's disease in patients with mild cognitive impairment. Mol Psychiatry. 9 (2004) 705-710;
Ref5: Blennow K, Zetterberg H. Fluid biomarker-based molecular phenotyping of Alzheimer's disease patients in research and clinical setting. Prog Mol Biol Transl Sci. 168 (2019) 3-23;
Ref6: Belnnow K, Zetterberg H. The past and future of Alzheimer's disease fluid biomarkers. J Alzheimers Dis. 63 (3) (2018) 1125-1140;
Ref7: Kylkilahti T M, Berends E, Ramos M, et al. Achieving brain clearance and preventing neurodegenerative diseases-A glymphatic perspective. J Cereb Blood Flow Metab (2021) 41(9):2137-2149;
Ref8: Tononi G, Cirelli C. Sleep and synaptic down-selection. Eur J Neurosci (2020) 51(1):413-421;
Ref9: Tononi G, Cirelli C. Sleep and the price of plasticity: from synaptic and cellular homeostasis to memory consolidation and integration. Neuron (2014) 81(1):12-34;
Ref10: Ju Y E, Lucey B P, Holtzman D M. Sleep and Alzheimer disease pathology—a bidirectional relationship. Nat Rev Neurol (2014) 10(2):115-119;
Ref11: Holth J K, Patel T K, Holtzman D M. Sleep in Alzheimer's disease-beyond amyloid. Neurobiol Sleep Circadian Rhythms (2017) 2:4-14;
Ref12: Holth J K, Fritschi S K, Wang C, et al. The sleep-wake cycle regulates brain interstitial fluid tau in mice and CSF tau in humans. Science (2019) 363(6429):880-884;
Ref13: Poe G R, Walsh C M, Bjorness T E. Both duration and timing of sleep are important to memory consolidation. Sleep (2010) 33(10):1277-1278;
Ref14: Ju Y E, Lucey B P, Holtzman D M (2014) Sleep and Alzheimer disease pathology—a bidirectional relationship, Nat Rev Neurol 10, 115-119;
Ref15: Hung C M, Li Y C, Chen H J, et al. (2018) Risk of dementia in patients with primary insomnia: a nationwide population-based case-control study, BMC Psychiatry 18, doi: 10.1186/s12888-018-1623-0;
Ref16: Lutsey P L, Misialek J R, Mosley T H, et al. (2018) Sleep characteristics and risk of dementia and Alzheimer's disease: The Atherosclerosis risk in communities study, Alzheimers Dement 14, 157-166;
Ref17: Latreille V, Carrier J, Lafortune M, et al. Sleep spindles in Parkinson's disease may predict the development of dementia. Neurobiol Aging (2015) 36(2):1083-1090;
Ref18: Montplaisir J, Petit D, Masson H et al. Sleep and quantitative EEG in patients with progressive supranuclear palsy. Neurology (1997) 49(4):999-1003;
Ref19: Varga A W, Wohlleber M E, Giménez S, et al. (2016) Reduced slow-wave sleep is associated with high cerebrospinal fluid Aβ42 levels in cognitively normal elderly, Sleep 39, 2041-2048;
Ref20: Ju Y S, Ooms S J, Sutphen C, et al. (2017) Slow wave sleep disruption increases cerebrospinal fluid amyloid-βlevels, Brain 140, 2104-2111;
Ref21: Liguori C, Nuccetelli M, Sancesario G, et al. (2016) Rapid eye movement sleep disruption and sleep fragmentation are associated with increased orexin-A cerebrospinal fluid levels in mild cognitive impairment due to Alzheimer's disease, Neurobiol Aging 40, 120-126;
Ref22: Pase M P, Himali J J, Grima N A, et al. (2017) Sleep architecture and risk of incident dementia in the community, Neurology 89, 1244-1250;
Ref23: Coon E A, Cutsforth-Gregory J K, Benarroch E E. Neuropathology of autonomic dysfunction in synucleinopathies. Movement Disorders, 2018. 33(3): p. 349-358;
Ref24: Chen, Z., G. Li, and J. Liu. Autonomic dysfunction in Parkinson's disease: Implications for pathophysiology, diagnosis, and treatment. Neurobiology of Disease, 2020. 134: p. 104700;
Ref25: St. Louis E K, Boeve B F. REM sleep behavior disorder; diagnosis, clinical implications and future directions, Mayo Clin Proc. 2017; 92, 1723-1736;
Ref26: Lin Y Q, Chen S D. RBD: a red flag for cognitive impairment in Parkinson's disease? Sleep Med 2018; 44, 38-44;
Ref27: Postuma R B, Iranzo A, Hu M, et al. Risk and predictors of dementia and parkinsonism in idiopathic REM sleep behavior disorder: a multicentre study. Brain. 2019; 142:744-759;
Ref28: Mano M, Nomura A, Sasanabe R. Gender differences in REM sleep behavior disorder in Japanese populations: Polysomnography and sleep questionnaire study. J Clin Med. 2024; 13; 914;
Ref29: Chahine L M, Xie S X, Simuni T, et al. Longitudinal changes in cognition in early Parkinson's disease patients with REM sleep behavior disorder. Parkinsonism Relat Disord. 2016; 27:102-106;
Ref30: Di Folco C. Couronne R, Anulf I, et al. Charting the trajectories from isolated REM sleep behavior disorder to Parkinson's disease. Mov Disord. 2024:39(1):64-75;
Ref31: Galbiati A, Verga L E, Giora M, et al. The risk of neurodegeneration in REM sleep behavior disorder: A systematic review and meta-analysis of longitudinal studies. Sleep Med Rev. 2019; 43:37-46;
Ref32: Levendowski D J, Gamaldo C, St. Louis E, et al. Head position during sleep: potential implications for patients with neurodegenerative disease. J Alzheimers Dis. 2019; 67(2):631-638;
Ref33: Head position during sleep: Potential implications for patients with neurodegenerative disorders. Levendowski D J, Walsh C, Neylan T, et al. Sleep Advances. 2023; 4: DOI sleepadvances/zpad035.097;
Ref34: Comparison of sleep and wake EEG biomarkers in mild cognitive impairment and Alzheimer's disease dementia. Meeting of the AAIC, Amsterdam, Netherlands. 2023;
Ref35: Levendowski D J, Lee-Iannotti J K, Tsuang D, et al. Atypical N3 sleep: A biomarker for altered mental status in Lewy body disease? Sleep Advances. 2023; 4: DOI sleepadvances/zpad035.110;
Ref36: Levendowski D J, Walsh C M, Boeve B G, et al. Non-REM sleep with hypertonia in Parkinsonian Spectrum Disorders: A pilot investigation. Sleep Med. 2022; DOI: 10.1016/j.sleep.2022.09.25;
Ref37: System and Method for detecting prodromal risk an/or severity of a-synuclein neurodegenerative disease. Provisional patent application No. 62/908,359, filed Sep. 30, 2019;
Ref38: Levendowski D J, Neylan T C, Lee-Iannotti J, et al. The accuracy and reliability of sleep staging and sleep biomarkers in patients with isolated rapid eye movement sleep behavior disorder. Nat Sci Sleep. 2023; 15:323-331;
Ref39: Levendowski D J, Neylan T C, Walsh C M, et al. Proof-of-concept for characterization of neurodegenerative disorders using two non-REM biomarkers. Front Neurol. 2023; 14: fneur.2023.1272369;
Ref40: Cho Y, Levendowski D J, Walsh C M, et al. Autonomic dysregulation during sleep in Parkinsonian Spectrum disorders—a proof-of-concept. Parkinsonism Relat Disord. 2023; in press;
Ref41: Pulver R L, Kronberg E, Medenblik L M et al. Mapping sleep's oscillatory events as a biomarker of Alzheimer's disease. J Alzheimers Dis. 2023; 1-15. DOI alz. 13420;
Ref42: Levendowski D J, Berka C, Neylan T C, et al. A community-based survey of personal perspectives regarding prodromal sleep screening for neurodegenerative disorders. Sleep. 2023: 46(1): A396-397; and
Ref43: Levendowski D J, Berka C, Neylan T C, et al. Potential role of physicians in addressing the needs of those at-risk of a neurodegenerative disorder-A pilot survey. Sleep. 2023: 46(1): A401.

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

As used herein, the terms “comprising,” “comprise,” and “comprises” are open-ended. For instance, “A comprises B” means that A may include either: (i) only B; or (ii) B in combination with one or a plurality, and potentially any number, of other components. In contrast, the terms “consisting of,” “consist of,” and “consists of” are closed-ended. For instance, “A consists of B” means that A only includes B with no other component in the same context.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

What is claimed is:

1. A method of characterizing a neurodegenerative disorder (NDD), the method comprising using at least one hardware processor to:
acquire physiological data for a subject, wherein the physiological data are obtained while the subject is sleeping;
derive one or more sleep biomarkers based on the physiological data;
apply a classifier to the one or more sleep biomarkers, wherein the classifier outputs a risk probability for each of one or more neurodegenerative disorders;
assign a risk severity based on the risk probability for each of the one or more neurodegenerative disorders; and
generate a report that indicates the risk severity for the subject.

2. The method of claim 1, wherein the one or more sleep biomarkers comprise a measure of time spent in rapid eye movement (REM) sleep.

3. The method of claim 1, wherein the one or more sleep biomarkers comprise a measure of spindle activity.

4. The method of claim 3, wherein deriving the measure of spindle activity comprises:
detecting each sleep spindle by
identifying a spindle peak comprising a burst in both sigma-wave power and alpha-wave power, within the physiological data,
determining a start time and an end time of the sleep spindle around the spindle peak based on at least one first threshold,
determining a duration of the sleep spindle based on the start time and the end time, and
detecting the sleep spindle when the duration of the sleep spindle satisfies at least one second threshold; and
computing the measure of spindle activity based on the duration of each detected sleep spindle.

5. The method of claim 1, wherein the one or more sleep biomarkers comprise a measure of atypical slow-wave (AN3) sleep.

6. The method of claim 1, wherein the one or more sleep biomarkers comprise a measure of non-rapid eye movement (NREM) hypertonia (NRH).

7. The method of claim 6, wherein deriving the measure of NREM hypertonia comprises automatically detecting one or more episodes of NREM hypertonia by:
for each of a plurality of epochs, represented in the physiological data,
determining whether or not the epoch exhibits an abnormal sleep characteristic based on signals, in the physiological data, representing delta-wave power without ocular activity, theta-wave power, sigma-wave power, and electromyographic (EMG) power, and
determining whether or not a standard deviation of the EMG power satisfies a predefined threshold within a set of two or more epochs that includes the epoch;
connecting two or more of the plurality of epochs, that each exhibits the abnormal sleep characteristic and for which the standard deviation of the EMG power satisfies the predefined threshold within the set of two or more epochs that includes the epoch, into an NRH block;
extending one or more NRH blocks to include one or more surrounding epochs; and
excluding any NRH block that satisfies one or more exclusion criteria,
wherein the one or more episodes of NREM hypertonia consist of any non-excluded NRH blocks.

8. The method of claim 7, wherein determining whether or not the epoch exhibits an abnormal sleep characteristic comprises:
determining that the epoch does not exhibit the abnormal sleep characteristic when the delta-wave power without ocular activity exceeds a first threshold;
calculating a delta threshold based on a theta-EMG ratio of the theta-wave power and the EMG power;
calculating a theta threshold based on a delta-EMG ratio of the delta-wave power without ocular activity and the EMG power;
determining whether or not the delta-EMG ratio is within a first range based on the delta threshold;
determining whether or not the theta-EMG ratio is within a second range based on the theta threshold;
determining whether or not a sigma-EMG ratio of the sigma-wave power and the EMG power is within a third range;
determining that the epoch does not exhibit the abnormal sleep characteristic when either the delta-EMG ratio is not within the first range, the theta-EMG ratio is not within the second range, or the sigma-EMG ratio is not within the third range; and
determining that the epoch exhibits the abnormal sleep characteristic when the delta-EMG ratio is within the first range, the theta-EMG ratio is within the second range, and the sigma-EMG ratio is within the third range.

9. The method of claim 7, wherein the one or more exclusion criteria comprise one or more of a presence of the NRH block within a predefined time duration immediately following sleep onset, the presence of the NRH block within a predefined time duration of uprightness, or the NRH block corresponding in time to a skin-electrode impedance greater than a predefined threshold impedance.

10. The method of claim 1, wherein the one or more sleep biomarkers comprise a measure of autonomic activation.

11. The method of claim 1, wherein the one or more sleep biomarkers comprise relative theta-wave power.

12. The method of claim 1, wherein the one or more sleep biomarkers comprise a ratio of theta-wave power to alpha-wave power.

13. The method of claim 1, wherein the one or more sleep biomarkers comprise a measure of sleep efficiency.

14. The method of claim 1, wherein the one or more sleep biomarkers comprise sleep duration in a supine position.

15. The method of claim 1, wherein the one or more sleep biomarkers comprise a measure of electroencephalographic (EEG) slowing.

16. The method of claim 1, wherein the one or more sleep biomarkers comprise a measure of rapid eye movement (REM) sleep without atonia (RSWA) events.

17. The method of claim 16, wherein deriving the measure of RSWA events comprises:
detecting the RSWA events by, for each of a plurality of epochs, represented in the physiological data,
filtering an electromyographic (EMG) signal in the physiological data for the epoch,
extracting a measure of EMG power from the filtered EMG signal,
computing baseline EMG power during REM sleep, and
detecting the RSWA event based on the measure of EMG power and the baseline EMG power; and
computing the measure of RSWA events based on the detected RSWA events.

18. The method of claim 1, wherein the one or more sleep biomarkers comprise a pattern of oscillatory events.

19. The method of claim 1, wherein the one or more sleep biomarkers are derived further based on a health record of the subject.

20. The method of claim 1, wherein the one or more neurodegenerative disorders are a plurality of neurodegenerative disorders.

21. The method of claim 20, wherein the report comprises the risk severity, a two-way comparison of each pair of the plurality of neurodegenerative disorders, and an analysis of each of the one or more sleep biomarkers.

22. The method of claim 1, wherein the one or more sleep biomarkers are a plurality of sleep biomarkers.

23. A system comprising:
at least one hardware processor; and
software configured to, when executed by the at least one hardware processor,
acquire physiological data for a subject, wherein the physiological data are obtained while the subject is sleeping,
derive one or more sleep biomarkers based on the physiological data,
apply a classifier to the one or more sleep biomarkers, wherein the classifier outputs a risk probability for each of one or more neurodegenerative disorders,
assign a risk severity based on the risk probability for each of the one or more neurodegenerative disorders, and
generate a report that indicates the risk severity for the subject.

24. A non-transitory computer-readable medium having instructions stored thereon, wherein the instructions, when executed by a processor, cause the processor to:
acquire physiological data for a subject, wherein the physiological data are obtained while the subject is sleeping;
derive one or more sleep biomarkers based on the physiological data;
apply a classifier to the one or more sleep biomarkers, wherein the classifier outputs a risk probability for each of one or more neurodegenerative disorders;
assign a risk severity based on the risk probability for each of the one or more neurodegenerative disorders; and
generate a report that indicates the risk severity for the subject.
